SEQUENCE LISTING (10 sequences)

SEQ ID NO: 1 - DNA, 2588 bp, Lolium perenne
  misc_feature (1903)..(1903): Any nucleotide
ggcacgagaaggagatgaaagaaatcagacaacaaccttcttcttagccttcctcatccccacttctatctcttccttcagttgcagggagcaaggaggaagagctagcagcagcagttggcagagagagtgagcgaagggcaaagaagaagaagaaaggaacactgccaatccaaggaaaccaaagaggagaagctttgttcttgtgcttggagttggtgtaagagggtattaaggcgatggcatcgaactcatcggcagcagcggcggcggccttcttcgggatgagagatggggaccagcaagaccagatgaagccgctgataacacagcaccagcagctggcagcggtggcactgcccggcgccgcgcctgcggcgtccagccaacacggcgcaccgccggcggcggcgccaccggccaagaagaagaggactctaccagacccagacgcggaggtgatcgcgctttctcccaagacgctgatggcgactaaccggttcgtgtgcgaggtgtgccagaagggtttccagcgcgagcagaacctgcagctacaccgtcgcgggcacaacctcccctggaagctgaagcagaagaacccgaaccaggtgcagcggcgtcgcgtgtacctgtgcccggaggtgacgtgcgtccaccacgacccgtcgcgcgccctgggcgatctcaccgggatcaagaagcacttctgccgcaagcacggcgagaagaagtggaagtgcgacaagtgctccaagcgctacgccgtgcagtccgactggaaggcgcactccaagatctgcggcacacgcgagtaccgctgcgactgcggcaccctcttctccaggagggacagcttcatcacccacagggccttctgcgacgcactggcgcaggagagcgccaggctgccgccgacgagcctcagcaccctcaccagccacctctacggtgccaccaacgccggcaacatggcgctcagcctgtcccaggtgggatcccacctcaactccaccatgcagcacgacggcggccaccaccaccacccgtccccagacctcctccgcctcggtggcggcggcggcagcagcagcatcgccgcgcggctcgaccacctcctgtctccgacaggcgcgtccgcgttccgcgcgaatcagcagcagccccagccggccttcttcctcaacgcggctccggggcaggacttcggtgacgatcaggggggcaacggaccccactcttcatgcagtcaaagcccttccacggctcatgcagttccggaccttcagggcaacggcgcggcggccggccatgggctcttcaacctgggcttctttgccaacaacggcaatagctccgggtcaagtcacgagcatgcaagccagggcatgatgagcaacgatcagttcagcggcggagctggtggaggcggcaacggctctgaggtgtcggccgcggggatctttggcggcaactttgttggtggggatcacatggcgcaggctgggatgtacaacgaccaggcggctatgctgccgcagatgtctgccaccgcgctgctccagaaggccgcgcagatgggcgcgacctccagccccaacggcgcggcgtccatgttcaggggcttcgccggctcctcgccgcagctgcgacaggcggcacctcagcacatggaccagaacgaggcgaacctcaacgagctgatgaactctctggctgctggtggcggcgtcaatgccgccggaatgttcggcggcgccaacggtggccccggcgcggggatgtttgatcctaggatgtgcgacatggaccagcacgaggtgaagttcagccagggcggaggcggtgttggtgccaaccctcctcctggtggtggtggtggtggtgccgccgcgccgccaacatnacgcggacttcctcggcgtggcgaagcggcatcgtgcccgggatatcgactccaagaggtgaccacaaccaaagcagcagcgacatgagctccctggaggccgagatgaagtcggcttcgtcctacaacgggggccggatggcatgatcgagagcttacacaagccgtaagctcccatcatcacagtgagtctaagacttgaagcaacctggaattagcatgcatgcatatatgagactgagatgagtaaggttaattagcgagttagcaactaagttgatcgcatgagtatggtcgcatgcatgcatgggataaggctagctagctagctctccatggggccatgaagactggagtaccgtgaaagggagaggcctcctctgagactaattaagtaattagctagtgcgagtgaacgcatgcatgtgatggccctagcttggtcgtcgaggagttggtgtcagtgtcggtcctgttgtttattttccttccttgtaagtggatctttgctgggttaagtatagttctggagatagaaccaataaggctattttagttttgtagctagccgaaatgggatgttggggagctgaacttgcccctgaaactccttcttctgttccttttattacccttgtagcgaaagaatgctgctcttacagtgcaaaaaaaaaaaaaaaaaact

SEQ ID NO: 2 - protein, 583 aa, Lolium perenne
  MISC_FEATURE (555)..(555): Any amino acid
MASNSSAAAAAAFFGMRDGDQQDQMKPLITQHQQLAAVALPGAAPAASSQHGAPPAAAPPAKKKRTLPDPDAEVIALSPKTLMATNRFVCEVCQKGFQREQNLQLHRRGHNLPWKLKQKNPNQVQRRRVYLCPEVTCVHHDPSRALGDLTGIKKHFCRKHGEKKWKCDKCSKRYAVQSDWKAHSKICGTREYRCDCGTLFSRRDSFITHRAFCDALAQESARLPPTSLSTLTSHLYGATNAGNMALSLSQVGSHLNSTMQHDGGHHHHPSPDLLRLGGGGGSSSIAARLDHLLSPTGASAFRANQQQPQPAFFLNAAPGQDFGDDQGGNGPHSSCSQSPSTAHAVPDLQGNGAAAGHGLFNLGFFANNGNSSGSSHEHASQGMMSNDQFSGGAGGGGNGSEVSAAGIFGGNFVGGDHMAQAGMYNDQAAMLPQMSATALLQKAAQMGATSSPNGAASMFRGFAGSSPQLRQAAPQHMDQNEANLNELMNSLAAGGGVNAAGMFGGANGGPGAGMFDPRMCDMDQHEVKFSQGGGGVGANPPPGGGGGGAAAPPTXRGLPRRGEAASCPGYRLQEVTTTKAAAT

SEQ ID NO: 3 - DNA, 2220 bp, Lolium perenne
ggcacgagctcgtgccgaattcggcacgagaaaagtatgacaaggagatgaaagaaatcagacaacaaccttcttcttagccttcctcatccccacttctatctcttccttcagttgcagggagcaaggaggaagagctagcagcagcagttggcagagagagtgagcgaagggcaaagaagaagaagaaaggaacactgccaatccaaggaaaccaaagaggagaagctttgttcttgtgcttggagttggtgtaagagggtattaaggcgatggcatcgaactcatcggcagcagcggcggcggccttcttcgggatgagagatggggaccagcaagaccagatgaagccgctgataacacagcaccagcagctggcagcggtggcactgcccggcgccgcgcctgcggcgtccagccaacacggcgcaccgccggcggcggcgccaccggccaagaagaagaggactctaccagacccagacgcggaggtgatcgcgctttctcccaagacgctgatggcgactaaccggttcgtgtgcgaggtgtgccagaagggtttccagcgcgagcagaacctgcagctacaccgtcgcgggcacaacctcccctggaagctgaagcagaagaacccgaaccaggtgcagcggcgtcgcgtgtacctgtgcccggaggtgacgtgcgtccaccacgacccgtcgcgcgccctgggcgatctcaccgggatcaagaagcacttctgccgcaagcacggcgagaagaagtggaagtgcgacaagtgctccaagcgctacgccgtgcagtccgactggaaggcgcactccaagatctgcggcacacgcgagtaccgctgcgactgcggcaccctcttctccaggagggacagcttcatcacccacagggccttctgcgacgcactggcgcaggagagcgccaggctgccgccgacgagcctcagcaccctcaccagccacctctacggtgccaccaacgccggcaacatggcgctcagcctgtcccaggtgggatcccacctcaactccaccatgcagcacgacggcggccaccaccaccacccgtccccagacctcctccgcctcggtggcggcggcggcagcagcagcatcgccgcgcggctcgaccacctcctgtctccgacaggcgcgtccgcgttccgcgcgaatcagcagcagccccagccggccttcttcctcaacgcggctccggggcaggacttcggtgacgatcaggggggcaacggaccccattccttcatgcagtcaaaagccctttccacggcctcatgcagctcccggaccttcagggcaacggcgcgggcgggccgggcccatgggctcttcaacctgggcttctttgccaacaacggcaatagctccgggtcaagtcacgagcatgcaagccagggcatgatgagcaacgatcagttcagcggcggagctggtggaggcggcaacggctctgaggtgtcggccgcggggatctttggcggcaactttgttggtggggatcacatggcgcaggctgggatgtacaacgaccaggcggctatgctgccgcagatgtctgccaccgcgctgctccagaaggccgcgcagatgggcgcgacctccagccccaacggcgcggcgtccatgttcaggggcttcgccggctcctcgccgcagctgcgacaggcggcacctcagcacatggaccagaacgaggcgaacctcaacgagctgatgaactctctggctgctggtggcggcgtcaatgccgccggaatgttcggcggcgccaacggtggccccggcgcggggatgtttgatcctaggatgtgcgacatggaccagcacgaggtgaagttcagccagggcggaggcggtgttggtgccaaccctgctgctggtggtggtggtggtggtggcggcggcggcggcgacatgacgcgggacttcctcggcgtgggcggaggcggcatcgtgcgcgggatatcgactccaagaggtgaccacaaccaaagcagcagcggacatgagctccctggaggccgaaatgaagtcggcttcgtcctacaacgggggccggatggcatgatcgagagcttacccaagccgtaagctcccatcatcccagtgagtttaagacttgaagcaacctggaattagcatgcatgcatatatgagactgagatgagtaaggttaattagcgagttagca

SEQ ID NO: 4 - protein, 615 aa, Lolium perenne
MASNSSAAAAAAFFGMRDGDQQDQMKPLITQHQQLAAVALPGAAPAASSQHGAPPAAAPPAKKKRTLPDPDAEVIALSPKTLMATNRFVCEVCQKGFQREQNLQLHRRGHNLPWKLKQKNPNQVQRRRVYLCPEVTCVHHDPSRALGDLTGIKKHFCRKHGEKKWKCDKCSKRYAVQSDWKAHSKICGTREYRCDCGTLFSRRDSFITHRAFCDALAQESARLPPTSLSTLTSHLYGATNAGNMALSLSQVGSHLNSTMQHDGGHHHHPSPDLLRLGGGGGSSSIAARLDHLLSPTGASAFRANQQQPQPAFFLNAAPGQDFGDDQGGNGPHSFMQSKALSTASCSSRTFRATARAGRAHGLFNLGFFANNGNSSGSSHEHASQGMMSNDQFSGGAGGGGNGSEVSAAGIFGGNFVGGDHMAQAGMYNDQAAMLPQMSATALLQKAAQMGATSSPNGAASMFRGFAGSSPQLRQAAPQHMDQNEANLNELMNSLAAGGGVNAAGMFGGANGGPGAGMFDPRMCDMDQHEVKFSQGGGGVGANPAAGGGGGGGGGGGDMTRDFLGVGGGGIVRGISTPRGDHNQSSSGHELPGGRNEVGFVLQRGPDGMIESLPKP

SEQ ID NO: 5 - DNA, 2474 bp, Lolium perenne
  misc_feature (571)..(571): Any nucleotide
gtcaagggcgagcgagaagaccaccacagctagcgaagcagcgcaagatcagatagataagagagacacgcaggtgaaaggaaatttgctagccgaaggctagcctacaaaaagggaaagctttgcctgcttccggcgagagtagctagcttcaacggcgatggcgtccaactcatcggcggcggctgtggcggcgttgtttggaattagggatggccaccagcaggaccagatcaagccgctgcttccgctgcagcagcagcaccaccagcccgccctcgcgccgcccagcgcggtggcggcgggaccggaccagccggctgcggcggtgccaccggtgaagaagaagagaaccatgcctgaccctgacgctgaggtgatcgcgctgtcgcccaagacgctgatggcgacgaaccggttcgtctgcgaggtgtgcaacaaggggttccagcgggagcagaacctgcagctgcaccggcgcggccacaacctgccgtggaagctgaagcagaaggaccccaaccaggtgcagcggcggcgggtgtacctctgcccggagccgacgtgcgtncaccacgagcccggccgcgcgctgggcgaccacaccggcatcaagaagcacttctgccgcaagcacggcgagaagaaatggaagtgcgacaagtgcgccaagcgctacgccgtgcagtcggactggaaggcgcactccaaggtctgcggcacccgcgagtaccgctgcgactgcggcaccctattctcccggagggacagcttcatcacccacagggccttctgcgacgccctcacccaggagagcgcgcgcctggcacccgccgcccacctctacggcggcgccacggccgctgccaacatggcgctcagcctctctcaggtgggatcctccttccaggacgtccacggccagtaccaccagcaggcatcctcggacctcctccgcttcggcggcggtggcggcggcggcggcatggctgcccgcctcgaccacctcctctcgtcgtccaccttccgcaacctgccgcctccccaagcaccggcgccgttccacctcggccagacgcagcaggagttcggcgacgggaacaacaacggcccgcacgcgttcctgcagggcaagccgttccacggcctcatgcagctcccggacctccagggcaacggctccgggggcacggcgtcgtcggctcccggtctcttcaacctcggcggctacatcgccaacaagcgccaacagctccggcggtacctcagccacggccacgccagccaggggcacatggccaacaatgaccagatcagtgaaggagccggtggtgccggttctgagaactccggcgcggcgttcttcaacgccgcggccggtgggaacttctccggtggcgaccaccaccaggttgttgctcccgctgggatgtacaacgagcagcaggccatggcgatgctgccgcagatgtcggccacggccctgctccagaaggcggcacagatgggctccagcaccagcgcggacggcggcggctcctcctccatattcagcggtttcctgggccagtccgaccagcagggtcgcgcgcccagcatggtggaccagggaatgcatctccagagcctgatgaactcgctggccggcgggagcaacggaggcgggatctttggaggcggcggcaacggccggggtatgattgatccgagactgtatgacatggaccagcacgaggtgaaatttagccagcagggccgtgggggcagcggcggcggcggtggagacgtgacaagggactttctcggcgttggtggcagaggggacgtgatcagagggatgtcagtggcaagaggggagcaccacagcggcggcggcggcgacatgagcttcttggaggccgagatgaagtcggcctcgtcgcccttcaatggaggcaggatgcagtgatgatgagcttaattttctttagaaaggcatgaagctggcttaagttcgttccgatcgagtcgtgaacctgagaaccatatgcatggaagaagctgtttacttacaactttgtttacatgtcagcacaaatgttttgacttctgggagcaaggttggaagggtatgcatcaatgcatgagcttcttcttccaccaattcaagaaaggtaaaacgactcaaatcattagactagatcttatatttagcggcagtcttcctgtcaatgttttgttaccgcagttgaatattattaaatgtaattggattaattcatgccggctagggagagatcgttgcatataccgtaaggtttttgtagctggcaatgtgagggatggttagaacttgcgcaccccaaactcaatctttttttcttttcttacaaataatgacattgctgtagcccgaatgggtggtatgaattttctttttccgtaaaaaaaaaaaaaaaaaaaaaaaaactcgagactagttctcgtgccgaatt

SEQ ID NO: 6 - protein, 595 aa, Lolium perenne
MASNSSAAAVAALFGIRDGHQQDQIKPLLPLQQQHHQPALAPPSAVAAGPDQPAAAVPPVKKKRTMPDPDAEVIALSPKTLMATNRFVCEVCNKGFQREQNLQLHRRGHNLPWKLKQKDPNQVQRRRVYLCPEPTCVHHEPGRALGDHTGIKKHFCRKHGEKKWKCDKCAKRYAVQSDWKAHSKVCGTREYRCDCGTLFSRRDSFITHRAFCDALTQESARLAPAAHLYGGATAAANMALSLSQVGSSFQDVHGQYHQQASSDLLRFGGGGGGGGMAARLDHLLSSSTFRNLPPPQAPAPFHLGQTQQEFGDGNNNGPHAFLQGKPFHGLMQLPDLQGNGSGGTASSAPGLFNLGGYIANKRQQLRRYLSHGHASQGHMANNDQISEGAGGAGSENSGAAFFNAAAGGNFSGGDHHQVVAPAGMYNEQQAMAMLPQMSATALLQKAAQMGSSTSADGGGSSSIFSGFLGQSDQQGRAPSMVDQGMHLQSLMNSLAGGSNGGGIFGGGGNGRGMIDPRLYDMDQHEVKFSQQGRGGSGGGGGDVTRDFLGVGGRGDVIRGMSVARGEHHSGGGGDMSFLEAEMKSASSPFNGGRMQ

SEQ ID NO: 7 - DNA, 1887 bp, Lolium perenne
  misc_feature (1712)..(1712): Any nucleotide
tcggcacgagtgcacatgcaccatcgccccgcacggccgcgagatctgatcgattttgcagcggctagctgaggattccgtggaaatttcagcagcgagctggtggatttttgtagctagctggggtcgatcgagatgatgctcaaggatctggcggcaattcagcagcagcagcagcagcagctggccctggccgcggcggcggacgagaacatgtccaacctcacctccgcgtccggcgaccagaccagcgtctcctcccaccctctcccgcctccttccaagaggaagcgcagcctcccgggaaacccagaccccgacgcggaggtgatcgcgctgtcgccgcggtcgctcatggccacgaaccgctacgtgtgcgagatctgcggcaagggcttccagcgggaccagaacctgcagctgcaccgccgcggccacaacctgccctggaagctcaagcagcgcaaccccaacgaggtggtgcgcaagaaggtctacgtctgcccggagcccggctgggtgcaccacgaccgcgcccgcgcgctcggcgacctcacggggatcaagaagcacttcagccgaaagcacggcgagaagaagtggaagtgcgacaagtgcgccaagcgatacgccgtgcagtccgactggaaggcgcactccaaggtctgcggcaccagggagtaccgctgcgactgcggcaccctcttctcaaggcgggacagcttcatcacgcacagggctttctgcgacgcgctggcggaggagagcgccagggccgtcgcggtggatccggggatgctctactctcacagcggcggcggcagcgcggggtttcagatgccggccgtcatggacgcgtcgcacccgctgggcgccgggcacgggctcatccaagaactgtgcctcaagagggagcagcagcagcaacatcaacaacagttcgcgcagccgtggctatccgagcagcagcagatggagatggccagcgagggcgcccccccggggatgtttggcacggcgaggatggaccaggagttcaacgggagcagcacgccggagagcagcacgcagccggcgggcatgggcttcgcgtccttctcgtcgcccgcggcggcggggccgtccgcctccgggtccacgcacatgtcagccaccgcgctgctccagaaggcggcgcagatgggcgcgacgctgagccgtccgtcgggccagggccagatggcgccgagtaccctcagcagcagcagcgttggcggcgccgccaacaataatgcaccggctgccgctactaccactaacagcgcgacgacaagcactgccatcggcgctggattcgcgcacacgttcgaggcgccagcgcacttcggggtgcaccagagatccaattccagcagtcgcaatgccggcaatggcgccaccggggccggaggtggtgcaatgccgggggcggcaacgacggccaaacgagggacttcctggggctgcgggcattctcccacggcgacatactcagcatggcagggttcgatccctgcatgcccacctcggcctctgcgtccgcggcagcgtacgatcagcaagggcaccagagcaccgagccatggcacggctagcaaggtacagtactagtgatgaacatagctatcagctggctagctcgccatcgatgagctgaaccgtgtcaattcgatgcaccatgccatggcctaatnttcatcgcctctggatgcccagcattgctctcgatcatatatgtcaagtttctcatggggtaagtggtcgatatccagttcgtttttgttgcacgccactcagtttgatcttcttttcggaggaaataatgaactgttcaaattcatgtaaaaaaaaaaaaaaaaaaaaaactcg

SEQ ID NO: 8 - protein, 497 aa, Lolium perenne
MMLKDLAAIQQQQQQQLALAAAADENMSNLTSASGDQTSVSSHPLPPPSKRKRSLPGNPDPDAEVIALSPRSLMATNRYVCEICGKGFQRDQNLQLHRRGHNLPWKLKQRNPNEVVRKKVYVCPEPGWVHHDRARALGDLTGIKKHFSRKHGEKKWKCDKCAKRYAVQSDWKAHSKVCGTREYRCDCGTLFSRRDSFITHRAFCDALAEESARAVAVDPGMLYSHSGGGSAGFQMPAVMDASHPLGAGHGLIQELCLKREQQQQHQQQFAQPWLSEQQQMEMASEGAPPGMFGTARMDQEFNGSSTPESSTQPAGMGFASFSSPAAAGPSASGSTHMSATALLQKAAQMGATLSRPSGQGQMAPSTLSSSSVGGAANNNAPAAATTTNSATTSTAIGAGFAHTFEAPAHFGVHQRSNSSSRNAGNGATGAGGGAMPGAATTAKRGTSWGCGHSPTATYSAWQGSIPACPPRPLRPRQRTISKGTRAPSHGTASKVQY

SEQ ID NO: 9 - protein, 509 aa, potato
MSIVTCEKAAASLSSSSNMNNDTNGAFCYTPQHQLVTPQYQNPPQQIKKKRNQPGNPDPEAEVIALSPKTLVAANRFFCEICNKGFQRDQNLQLHRRGHNLPWKLKKRENKEVVRKKVYICPESSCVHHDPSRALGDLTGIKKHFSRKHGEKKWKCEKCSKRYAVQSDCKAHFKTCGTREYKCECGTIFSRRDSFITHRAFCETLAMESARSVINGRNPTIFSPQLNLQFQQPHFFNSHEQIQATTFPMKKEQQSSDFRHIEIPPWLITTNSQPFQLGAINHGPSPRSNFSSSSIFPATTRLDQQYTQSGHKDLNLHHPNPNLRGPTLGYDSTGESGAVSPVHISATRLLQKAAQFGATISNKASAVTATAAYTGTVKIPHNTHVSVTSTDSATKQTHQKLSSREDLTSITGPANISGIMTSFSNGFDGSTMFEDAILFGGFNNLNSKKEDEEEDQQLYFNGSMNEEDHILTKDFLGLKPLSHTDDIFNIAALVNTEPHHFKNHKTWQS

SEQ ID NO: 10 - protein, 436 aa, maize
MQMMMLSDLSSDDHEATGSSSYGGDMASYALSPLFLAPAASATAPLPPPPQPPAEELTNKQAAGGGKRKRSQPGNPDPGAEVIALSPRTLVATNRFVCEICNKGFQRDQNLQLHRRGHNLPWKLRQRSSLVVPSSSAAAGSGGRQQQQQGEAAPTPPRKRVYVCPEPTCVHHDPARALGDLTGIKKHFSRKHGEKRWCCERCGKRYAVQSDWKAHVKGCGTREYRCDCGILFSRKDSLLTHRAFCDALAEESARLLAAAANNGSTITTTSSSNNNDLLNASNNITPLFLPFASSPPPVVVAAAQNPNNTLFFLHQELSPFLQPRVTMQQQPSPYLDLHMHVDASIVTTTGGLADGTPVSFGLALDGSVATVGHRRLTRDFLGVDGGGRQVEELQLPLCATAAAAGASRTASCATDLTRQCLGGRLPPVNETWSHNF

(19) United States
(12) Patent Application Publication
(10) Pub. No.: US 2004/0098767 A1
(43) Pub. Date: May 20, 2004

(54) Manipulation of plant life cycles and/or growth phases

(76) Inventors: German Spangenberg, Bundoora (AU); Bing Liu, Altrinham (GB); Dirk Truman, Croydon (AU)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/416,316

(22) PCT Filed: Nov. 7, 2001
(86) PCT No.: PCT/AU01/01432

(30) Foreign Application Priority Data
Nov. 8, 2000 (AU) PR 1313

(51) Int. Cl. A01H 1/00; C12N 15/82; C07H 21/04; A01H 5/00

(52) U.S. Cl. 800/320; 435/468; 435/320.1; 800/287; 536/23.2; 435/419

(57) ABSTRACT

The present invention relates to nucleic acids and nucleic acid fragments encoding the protein Indeterminate1 (ID1) from rye grass (Lolium), especially perennial rye grass (Lolium penne), involved in the transition to flowering in plants, and the use thereof in the modification of plant life cycles and/or growth phases, flowering processes, flowering and plant architecture, and inflorescence and flower development.

The present invention relates to nucleic acids and nucleic acid fragments encoding amino acid sequences for proteins involved in the control of the transition to flowering in plants and the use thereof for the modification of plant life cycles and/or growth phases, flowering processes, flowering and plant architecture, and inflorescence and flower development.

Most plants have several growth phases. Following seed embryo germination, the plant apical meristem goes through a vegetative phase generating leaf primordia with axillary meristems. The axillary meristems will generate side branches or will rest dormant until apical dominance is removed. Upon receiving appropriate signals, the apical meristem switches to reproductive development (flowering). The switch is controlled by various physiological signals and genetic pathways that will coordinate flowering. The apical meristem switched from vegetative to reproductive phase will produce reproductive structures (inflorescences and flowers) instead of vegetative structures (leaves). This point is a critical developmental process in flowering plants.

The INDETERMINATE1 (id1) gene of maize controls the transition of flowering in this species by encoding a putative transcriptional regulator of flowering transition. An id1 mutation is the only mutation known to specifically and severely alter the ability of maize to undergo the transition to reproductive growth. Homozygous id1 maize mutants will produce many more leaves than wild-type maize plants. Maize id1 mutants remain in a prolonged vegetative growth state.

While nucleic acid sequences encoding some of the proteins involved in the control of plant life cycles and growth phases, flowering processes, flowering and plant architecture, and inflorescence and flower development have been isolated for certain species of plants, there remains a need for materials useful in the control of plant life cycles and growth phases, flowering processes, flowering and plant architecture, and inflorescence and flower development, in a wide range of plants, particularly in grasses and cereals including ryegrasses and fescues, and for methods for their use.

It is an object of the present invention to overcome, or at least alleviate, one or more of the difficulties or deficiencies associated with the prior art.

In one aspect, the present invention provides substantially purified or isolated nucleic acids and nucleic acid fragments encoding amino acid sequences for an ID1 protein from a ryegrass (Lolium) or fescue (Festuca) species, or a functionally active fragment or variant thereof.

The present invention also provides substantially purified or isolated nucleic acids and nucleic acid fragments encoding amino acid sequences for a class of proteins which are related to ID1. Such proteins are referred to herein as ID1-like.

The down-regulation or enhancement or ectopic expression or otherwise manipulation of id1 gene activity in grasses and cereals may alter plant life cycles and growth phases, for example it may alter the control of phase change, promote or reduce vegetative growth, delay or otherwise alter flowering, and/or alter floral organ and plant architecture e.g. vegetative-like inflorescences and flowers, enhanced branching, increased bushiness.

Manipulation of, for example, transition from vegetative phase to flowering phase or plant life cycles has significant consequences for a wide range of applications in plant production. For example, it has applications in delaying flowering in forage grasses and cereals thus reducing the formation of the less digestible stems and increasing herbage quality, in altering flowering time allowing early or late maturing grass and cereal crops, in delaying vegetative phase and thus increasing biomass production, in increasing branching and thus leading to enhanced bushiness, in altering plant size and leading to either higher or shorter plant stature, in blocking flowering and reducing the release of allergenic pollen, etc.

Methods of manipulating plant life cycles and growth phases, eg. the transition from the vegetative to the reproductive state, flowering and plant architecture in plants, including forage grasses and cereals, and grass species such as ryegrasses (Lolium species) and fescues (Festuca species), may facilitate the production of, for example, pasture grasses with enhanced or shortened or modified life cycles, enhanced or reduced or otherwise modified inflorescence and flower development, inhibited flowering (including non-flowering), modified flowering architecture (indeterminate and determinate), earlier or delayed flowering, enhanced or modified number of leaves, enhanced or reduced or otherwise modified number of reproductive shoots, enhanced persistence and improved herbage quality, enhanced seed and leaf yield, altered growth and development, leading to improved seed production, improved biomass production, improved pasture production, improved pasture quality, improved animal production and reduced environmental pollution (e.g. reduced pollen allergens, reduced nitrogenous waste).

The ryegrass (Lolium) or fescue (Festuca) species may be of any suitable type, including Italian or annual ryegrass, perennial ryegrass, tall fescue, meadow fescue and red fescue. Preferably the species is a ryegrass, more preferably perennial ryegrass (L. perenne). Perennial ryegrass (Lolium perenne L.) is a key pasture grass in temperate climates throughout the world.

The nucleic acid or nucleic acid fragment may be of any suitable type and includes DNA (such as cDNA or genomic DNA) and RNA (such as mRNA) that is single- or double-stranded, optionally containing synthetic, non-natural or altered nucleotide bases, and combinations thereof.

The term “isolated” means that the material is removed from its original environment (eg. the natural environment if it is naturally occurring). For example, a naturally occurring nucleic acid present in a living plant is not isolated, but the same nucleic acid separated from some or all of the coexisting materials in the natural system, is isolated. Such nucleic acids could be part of a vector and/or such nucleic acids could be part of a composition, and still be isolated in that such a vector or composition is not part of its natural environment.

Such nucleic acids or nucleic acid fragments could be assembled to form a consensus contig. As used herein, the term “consensus contig” refers to a nucleotide sequence that is assembled from two or more constituent nucleotide sequences that share common or overlapping regions of sequence homology. For example, the nucleotide sequence of two or more nucleic acids or nucleic acid fragments can be compared and aligned in order to identify common or overlapping sequences. Where common or overlapping sequences exist between two or more nucleic acids or nucleic acid fragments, the sequences (and thus their corresponding nucleic acids or nucleic acid fragments) may be assembled into a single contiguous nucleotide sequence.

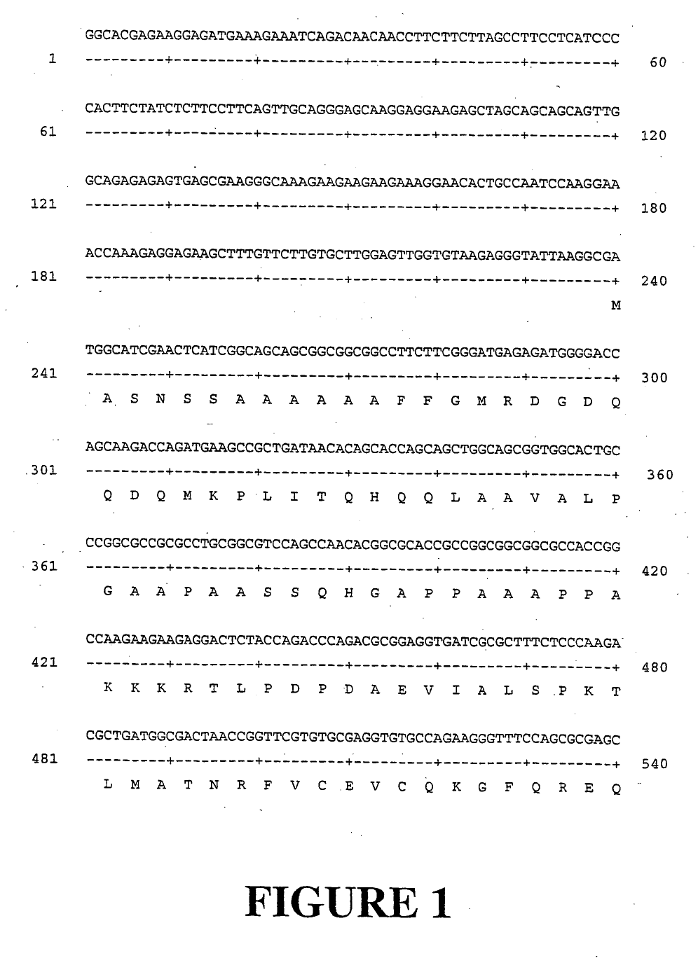

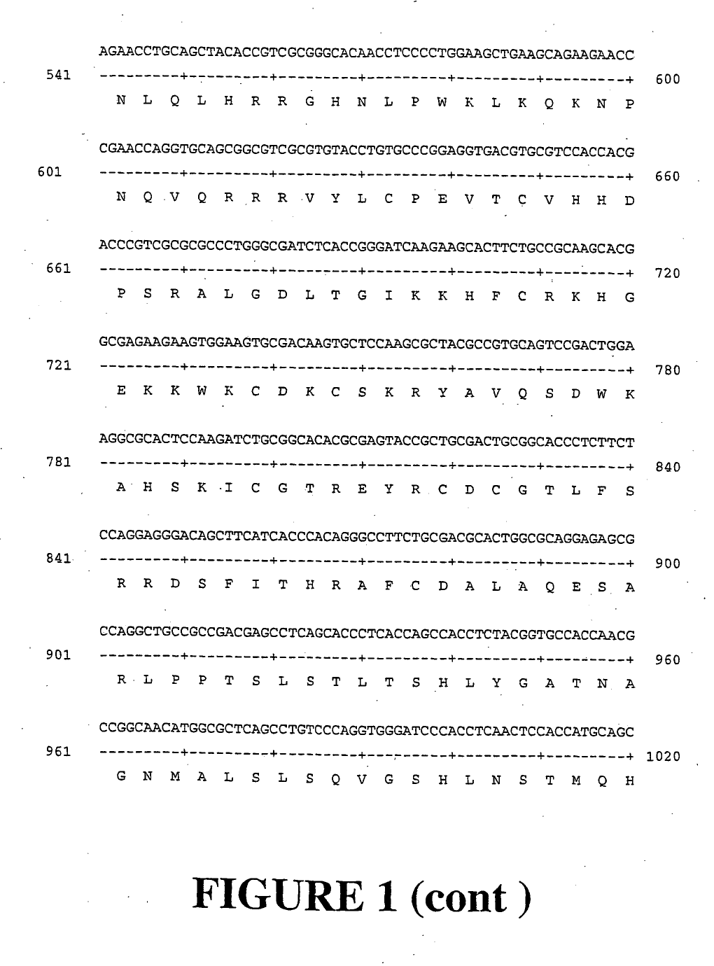

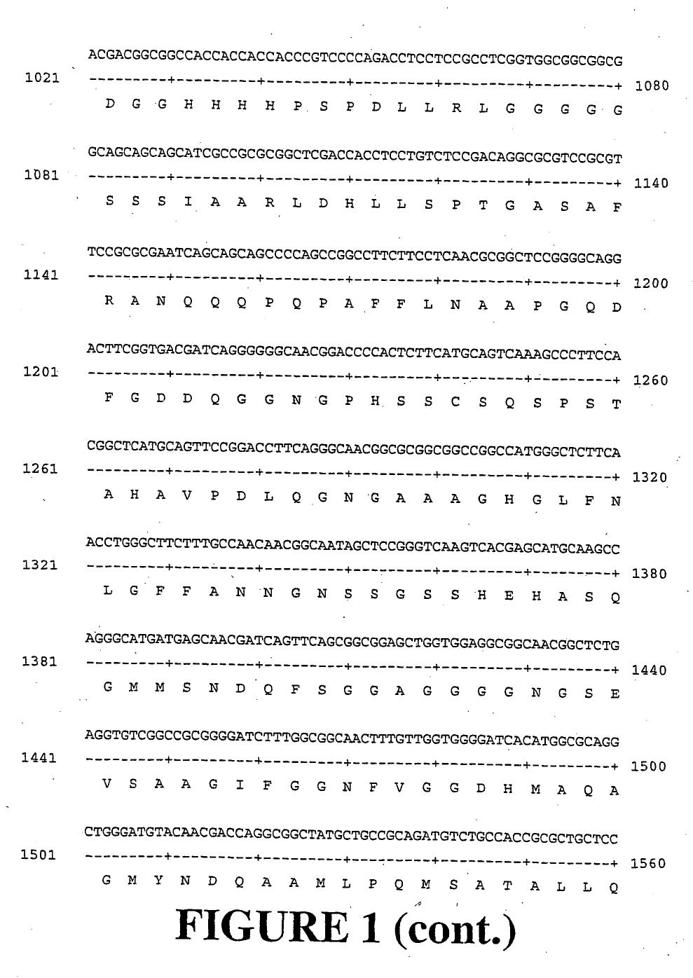

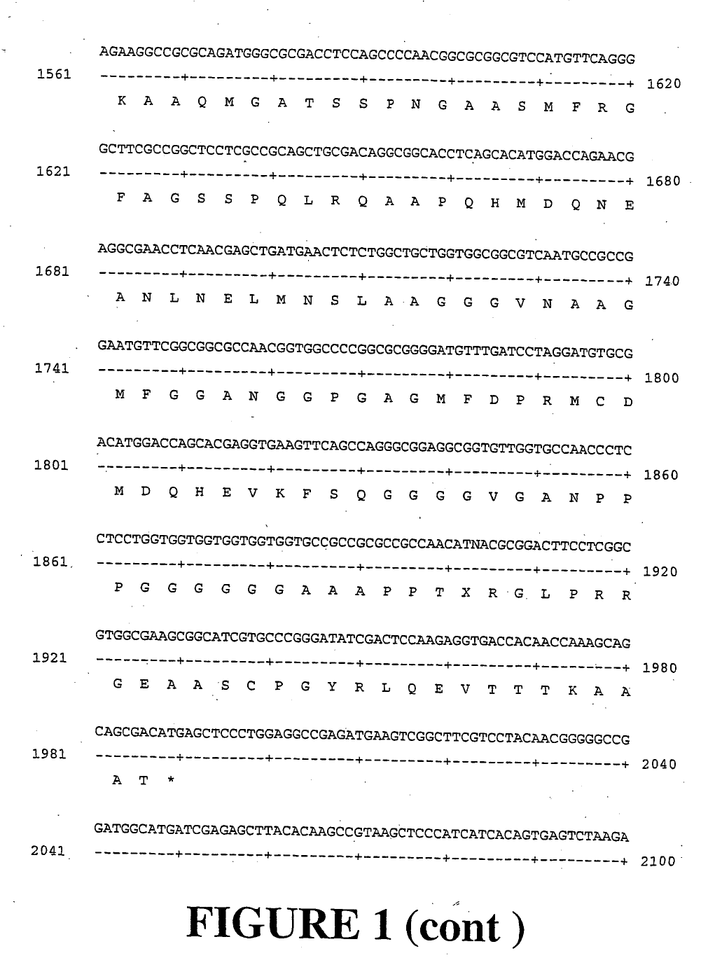

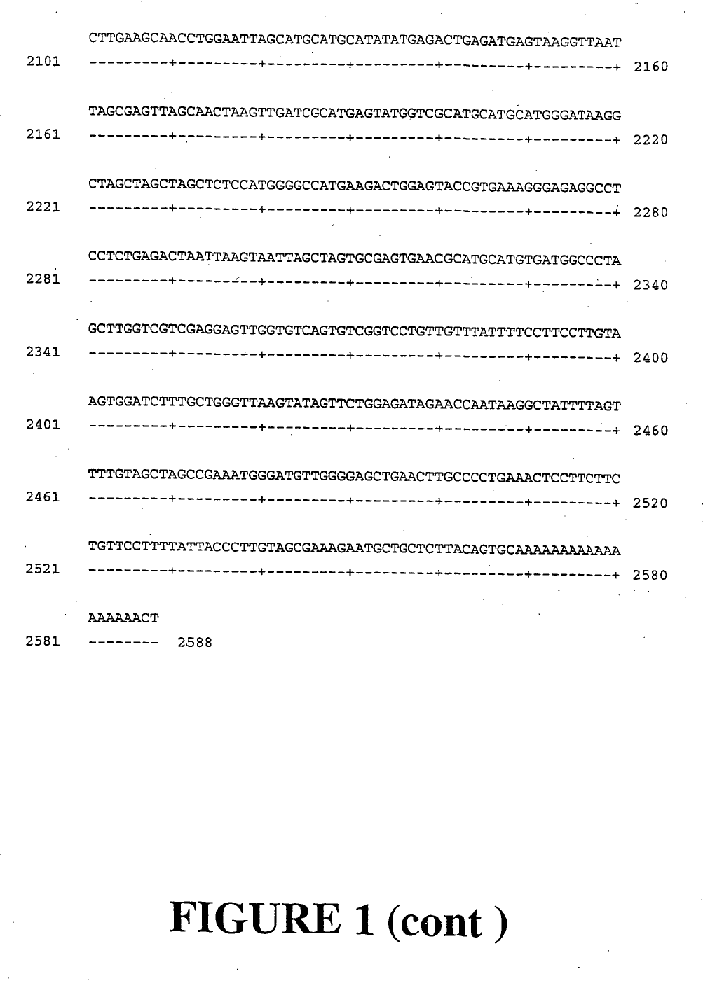

In a preferred embodiment of this aspect of the invention, the substantially purified or isolated nucleic acid or nucleic acid fragment encoding an ID1 or ID1-like protein includes a nucleotide sequence selected from the group consisting of (a) sequences shown in FIGS. 1, 2, 3, 4 and 5 hereto (Sequence ID Nos: 1, 3, 5 and 7); (b) complements of the sequences recited in (a); (c) sequences antisense to the sequences recited in (a) and (b); and (d) functionally active fragments and variants of the sequences recited in (a), (b) and (c).

By “functionally active” in relation to nucleic acids it is meant that the fragment or variant (such as an analogue, derivative or mutant) is capable of modifying the control of plant life cycles and/or growth phases, including flowering processes, and/or flowering or plant architecture in a plant. Such variants include naturally occurring allelic variants and non-naturally occurring variants. Additions, deletions, substitutions and derivatizations of one or more of the nucleotides are contemplated so long as the modifications do not result in loss of functional activity of the fragment or variant. Preferably the functionally active fragment or variant has at least approximately 80% identity to the relevant part of the above mentioned sequence, more preferably at least approximately 90% identity, most preferably at least approximately 95% identity. Such functionally active variants and fragments include, for example, those having nucleic acid changes which result in conservative amino acid substitutions of one or more residues in the corresponding amino acid sequence. Preferably the fragment has a size of at least 10 nucleotides, more preferably at least 15 nucleotides, most preferably at least 20 nucleotides.

The nucleic acids or nucleic acid fragments encoding at least a portion of proteins involved in the control of plant life cycles and/or growth phases, including flowering processes, and/or flowering or plant architecture have been isolated and identified. The nucleic acids and nucleic acid fragments of the present invention may be used to isolate cDNAs and genes encoding homologous proteins from the same or other plant species. Isolation of homologous genes using sequence-dependent protocols is well known in the art. Examples of sequence-dependent protocols include, but are not limited to, methods of nucleic acid hybridisation, and methods of DNA and RNA amplification as exemplified by various uses of nucleic acid amplification technologies (e.g. polymerase chain reaction, ligase chain reaction).

For example, genes encoding other proteins involved in the control of plant life cycles and/or growth phases, including flowering processes, and/or flowering or plant architecture, either as cDNAs or genomic DNAs, may be isolated directly by using all or a portion of the nucleic acids or nucleic acid fragments of the present invention as hybridisation probes to screen libraries from the desired plant employing the methodology well known to those skilled in the art. Specific oligonucleotide probes based upon the nucleic acid sequences of the present invention may be designed and synthesized by methods known in the art. Moreover, the entire sequences may be used directly to synthesize DNA probes by methods known to the skilled artisan such as random primer DNA labelling, nick translation, or end-labelling techniques, or RNA probes using available in vitro transcription systems. In addition, specific primers may be designed and used to amplify a part or all of the sequences of the present invention. The resulting amplification products may be labelled directly during amplification reactions or labelled after amplification reactions, and used as probes to isolate full length cDNA or genomic fragments under conditions of appropriate stringency.

In addition, short segments of the nucleic acids or nucleic acid fragments of the present invention may be used in amplification protocols to amplify longer nucleic acids or nucleic acid fragments encoding homologous genes from DNA or RNA. For example, the polymerase chain reaction may be performed on a library of cloned nucleic acid fragments wherein the sequence of one primer is derived from a nucleic acid or nucleic acid fragment of the present invention, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, those skilled in the art can follow the RACE protocol (Frohman et al. (1988) Proc. Natl. Acad Sci. USA 85:8998, the entire disclosure of which is incorporated herein by reference) to generate cDNAs by using PCR to amplify copies of the region between a single point in the transcript and the 3′ or 5′ end. Using commercially available 3′ RACE and 5′ RACE systems (BRL), specific 3′ or 5′ cDNA fragments may be isolated (Ohara et al. (1989) Proc. Natl. Acad Sci USA 86:5673; Loh et al. (1989) Science 243:217, the entire disclosures of which are incorporated herein by reference). Products generated by the 3′ and 5′ RACE procedures may be combined to generate full-length cDNAs.

In a second aspect of the present invention there is provided a substantially purified or isolated polypeptide from a ryegrass (Lolium) or fescue (Festuca) species, selected from the group consisting of ID1 and ID1-like proteins; and functionally active fragments and variants thereof.

The ryegrass (Lolium) or fescue (Festuca) species may be of any suitable type, including Italian or annual ryegrass, perennial ryegrass, tall fescue, meadow fescue and red fescue. Preferably the species is a ryegrass, more preferably perennial ryegrass (L. perenne).

In a preferred embodiment of this aspect of the invention, the substantially purified or isolated ID1 or ID1-like polypeptide includes an amino acid sequence selected from the group consisting of sequences shown in FIGS. 1, 2, 3, 4 and 6 hereto (Sequence ID Nos: 2, 4, 6 and 8) and functionally active fragments and variants thereof.

By “functionally active” in relation to polypeptides it is meant that the fragment or variant has one or more of the biological properties of the proteins ID1 or ID1-like. Additions, deletions, substitutions and derivatizations of one or more of the amino acids are contemplated so long as the modifications do not result in loss of functional activity of the fragment or variant. Preferably the functionally active fragment or variant has at least approximately 60% identity to the relevant part of the above mentioned sequence, more preferably at least approximately 80% identity, most preferably at least approximately 90% identity. Such functionally active variants and fragments include, for example, those having conservative amino acid substitutions of one or more residues in the corresponding amino acid sequence. Preferably the fragment has a size of at least 10 amino acids, more preferably at least 15 amino acids, most preferably at least 20 amino acids.

In a further embodiment of this aspect of the invention, there is provided a polypeptide recombinantly produced from a nucleic acid or nucleic acid fragment according to the present invention. Techniques for recombinantly producing polypeptides are well known to those skilled in the art.

Availability of the nucleotide sequences of the present invention and deduced amino acid sequences facilitates immunological screening of cDNA expression libraries. Synthetic peptides representing portions of the instant amino acid sequences may be synthesized. These peptides may be used to immunise animals to produce polyclonal or monoclonal antibodies with specificity for peptides and/or proteins including the amino acid sequences. These antibodies may be then used to screen cDNA expression libraries to isolate full-length cDNA clones of interest.

A genotype is the genetic constitution of an individual or group. Variations in genotype are important in commercial breeding programs, in determining parentage, in diagnostics and fingerprinting, and the like. Genotypes can be readily described in terms of genetic markers. A genetic marker identifies a specific region or locus in the genome. The more genetic markers, the finer defined is the genotype. A genetic marker becomes particularly useful when it is allelic between organisms because it then may serve to unambiguously identify an individual. Furthermore, a genetic marker becomes particularly useful when it is based on nucleic acid sequence information that can unambiguously establish a genotype of an individual and when the function encoded by such nucleic acid is known and is associated with a specific trait. Such nucleic acids and/or nucleotide sequence information including single nucleotide polymorphisms (SNP's), variations in single nucleotides between allelic forms of such nucleotide sequence, can be used as perfect markers or candidate genes for the given trait.

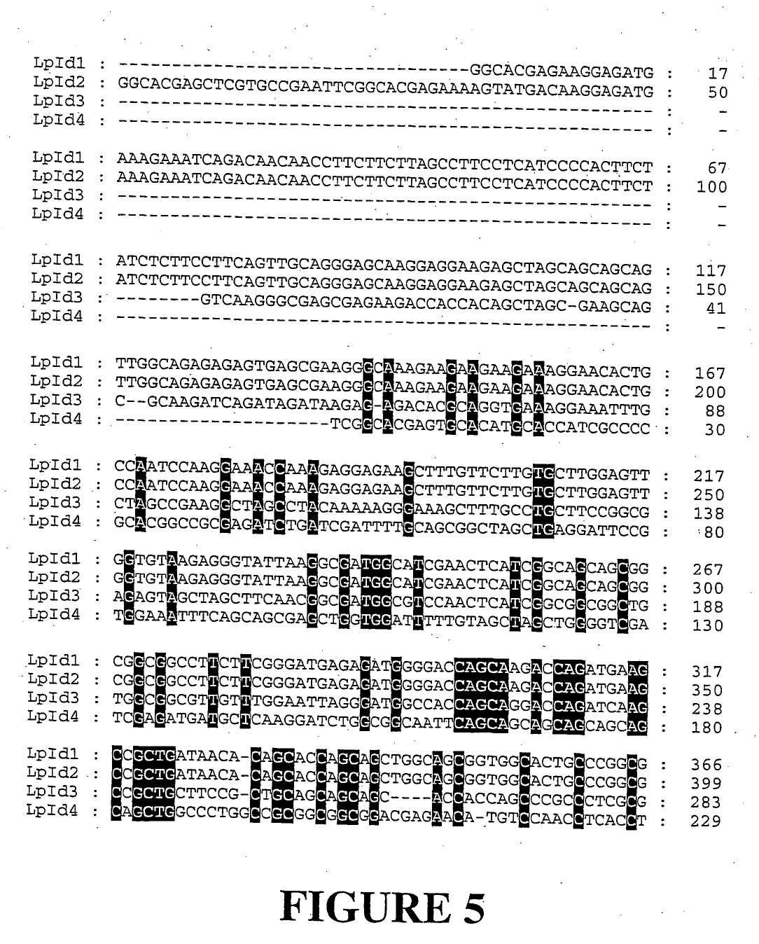

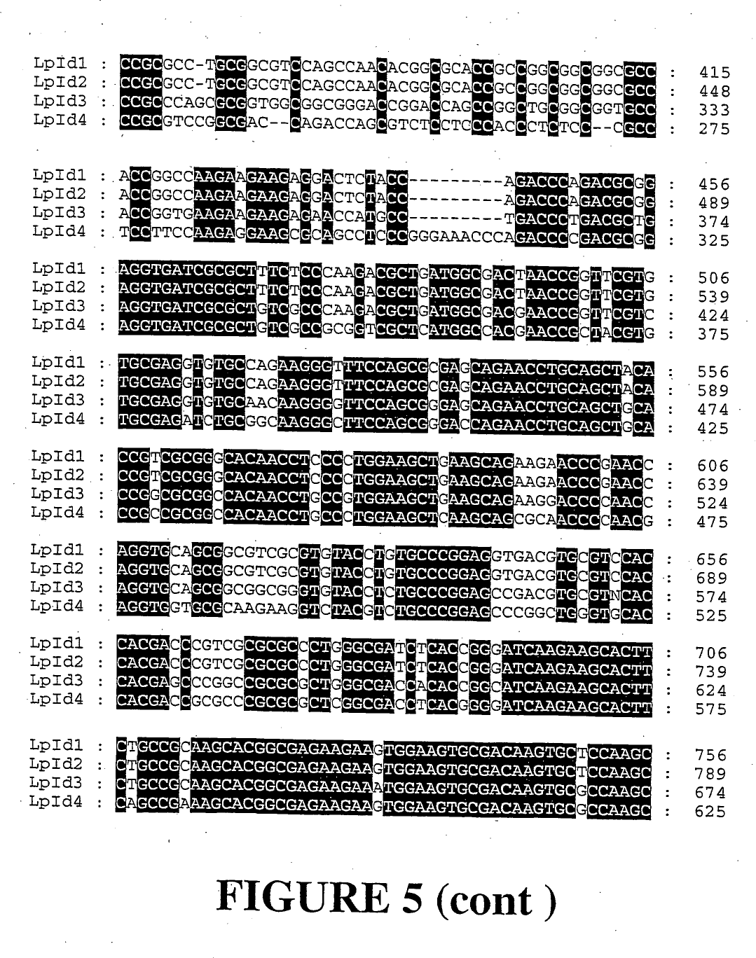

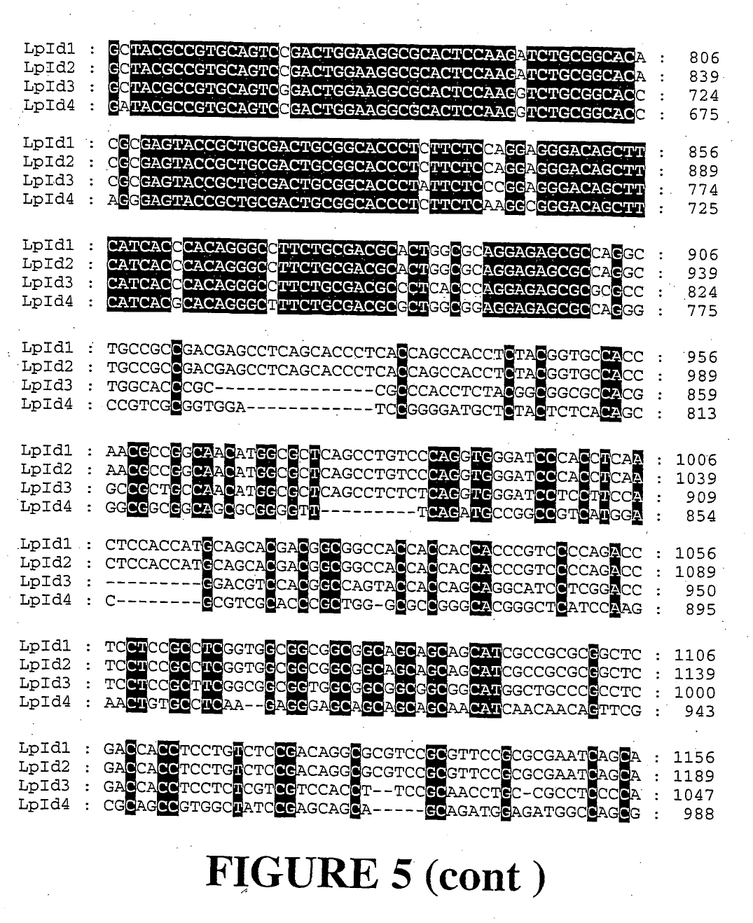

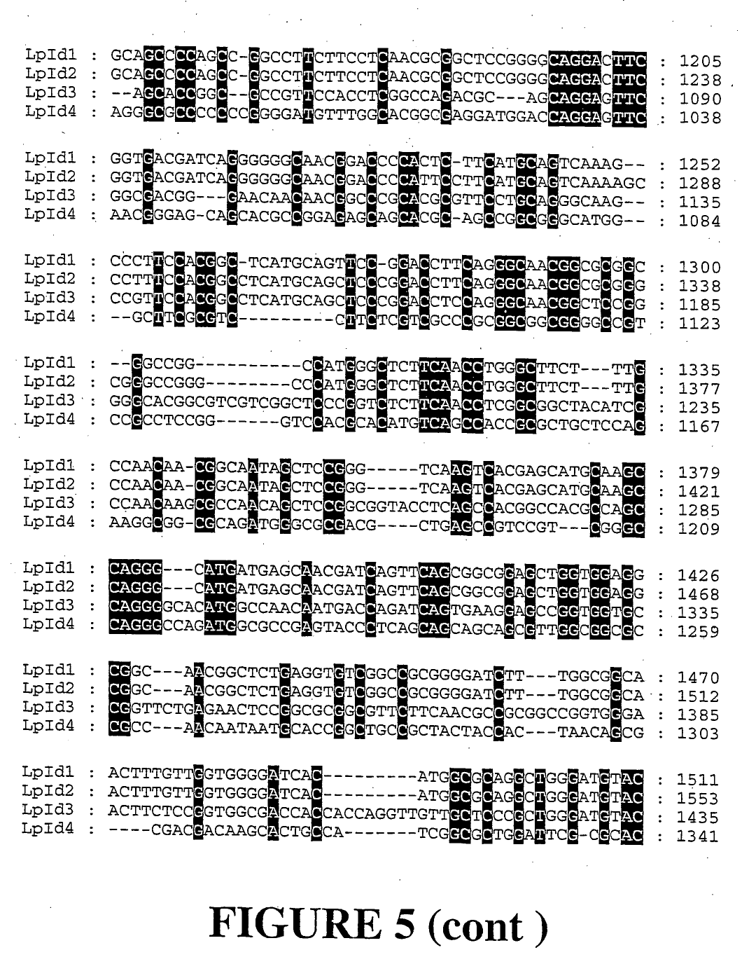

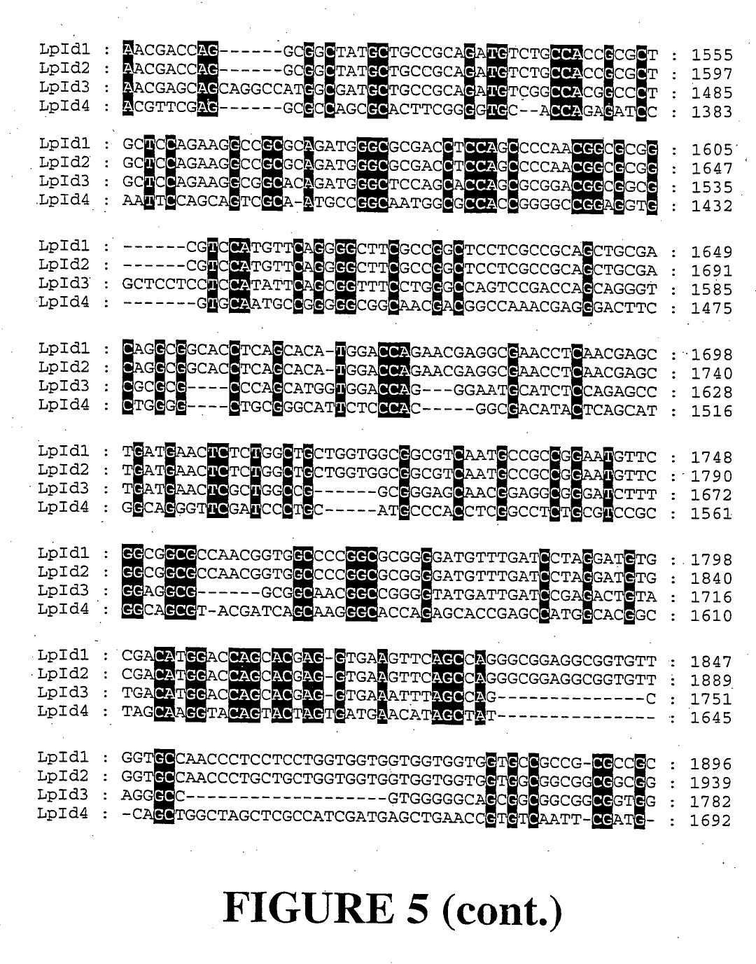

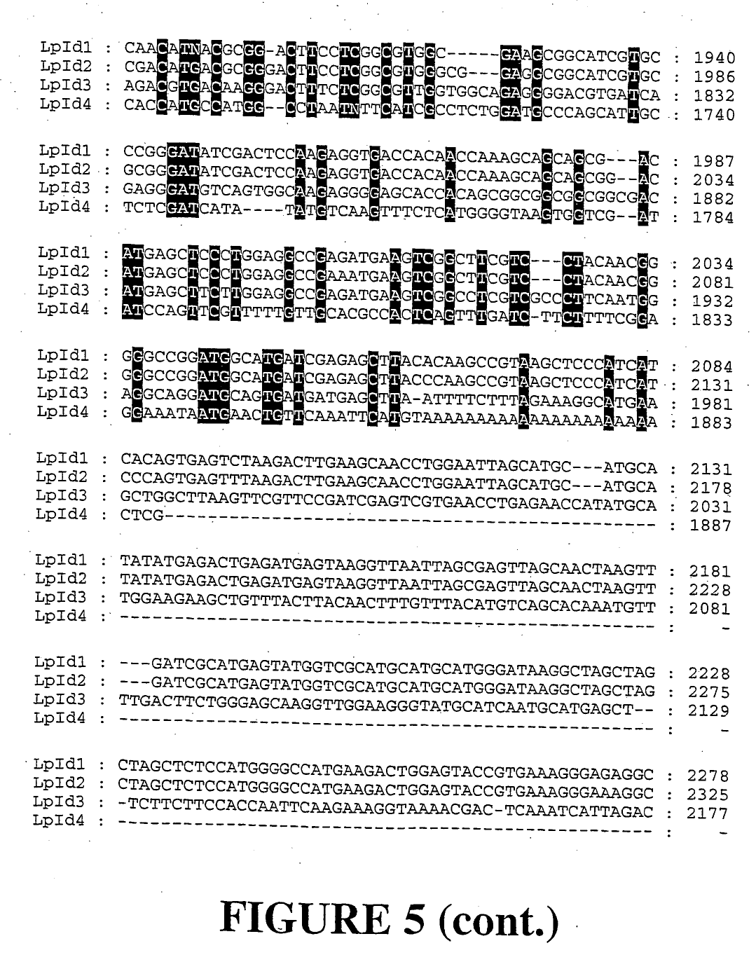

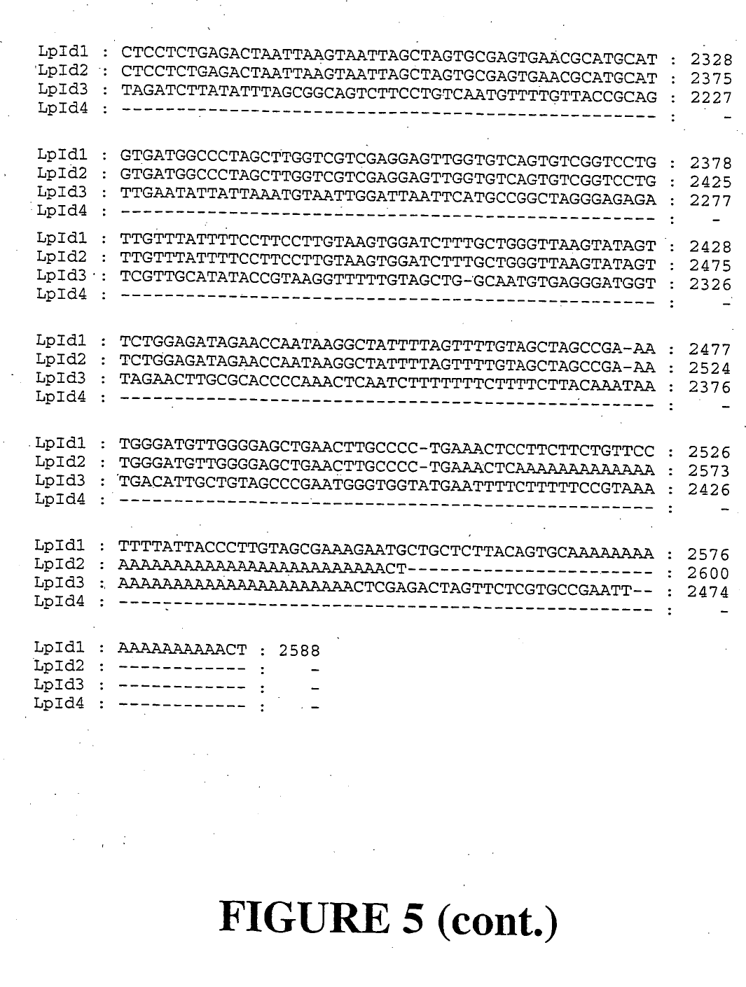

Applicants have identified a number of SNP's of the nucleic acids and nucleic acid fragments of the present invention. These are present in FIG. 5 (Sequence ID Nos: 1, 3, 5 and 7), which shows multiple alignments of nucleotide sequences of id1 nucleic acids of the present invention.

Accordingly, in a further aspect of the present invention, there is provided a substantially purified or isolated nucleic acid or nucleic acid fragment including a single nucleotide polymorphism (SNP) from a nucleic acid or nucleic acid according to the present invention, or complements or sequences antisense thereto, and functionally active fragments and variants thereof.

In a still further aspect of the present invention there is provided a-method of isolating a nucleic acid or nucleic acid fragment of the present invention including a single nucleotide polymorphism (SNP), said method including sequencing nucleic acid fragments from a nucleic acid library.

The nucleic acid library may be of any suitable type and is preferably a cDNA library.

The nucleic acid or nucleic acid fragment may be isolated from a recombinant plasmid or may be amplified, for example using polymerase chain reaction.

The sequencing may be performed by techniques known to those skilled in the art.

In a still further aspect of the present invention, there is provided use of nucleic acids or nucleic acid fragments of the present invention including SNPs, and/or nucleotide sequence information thereof, as molecular genetic markers.

In a still further aspect of the present invention there is provided use of a nucleic acid according to the present invention, and/or nucleotide sequence information thereof, as a molecular genetic marker.

More particularly, nucleic acids or nucleic acid fragments according to the present invention and/or nucleotide sequence information thereof may be used as a molecular genetic marker for quantitative trait loci (QTL) tagging, QTL mapping, DNA fingerprinting and in marker assisted selection, particularly in ryegrasses and fescues. Even more particularly, nucleic acids or nucleic acid fragments according to the present invention and/or nucleotide sequence information thereof may be used as molecular genetic markers in forage and turf grass improvement, e.g. tagging QTLs for herbage quality traits, flowering intensity, flowering time, number of tillers, leafiness, bushiness, seasonal growth pattern, herbage yield, flower architecture, plant stature. Even more particularly, sequence information revealing SNPs in allelic variants of the nucleic acids or nucleic acid fragments of the present invention and/or nucleotide sequence information thereof may be used as molecular genetic markers for QTL tagging and mapping and in marker assisted selection, particularly in ryegrasses and fescues.

In a still further aspect of the present invention there is provided a construct including a nucleic acid or nucleic acid fragment according to the present invention.

The term “construct” as used herein refers to an artificially assembled or isolated nucleic acid molecule which includes the gene of interest. In general a construct may include the gene or genes of interest, a marker gene which in some cases can also be the gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

In a still further aspect of the present invention there is provided a vector including a nucleic acid or nucleic acid fragment according to the present invention.

The term “vector” as used herein includes both cloning and expression vectors. Vectors are often recombinant molecules including nucleic acid molecules from several sources.

In a preferred embodiment of this aspect of the invention, the vector may include a regulatory element such as a promoter, a nucleic acid or nucleic acid fragment according to the present invention and a terminator; said regulatory element, nucleic acid or nucleic acid fragment and terminator being operatively linked.

By “operatively linked” is meant that said regulatory element is capable of causing expression of said nucleic acid or nucleic acid fragment in a plant cell and said terminator is capable of terminating expression of said nucleic acid or nucleic acid fragment in a plant cell. Preferably, said regulatory element is upstream of said nucleic acid or nucleic acid fragment and said terminator is downstream of said nucleic acid or nucleic acid fragment.

The vector may be of any suitable type and may be viral or non-viral. The vector may be an expression vector. Such vectors include chromosomal, non-chromosomal and synthetic nucleic acid sequences, eg. derivatives of plant viruses; bacterial plasmids; derivatives of the Ti plasmid from Agrobacterium tumefaciens, derivatives of the Ri plasmid from Agrobacterium rhizogenes; phage DNA; yeast artificial chromosomes; bacterial artificial chromosomes; binary bacterial artificial chromosomes; vectors derived from combinations of plasmids and phage DNA. However, any other vector may be used as long as it is replicable, integrative or viable in the plant cell.

The regulatory element and terminator may be of any suitable type and may be endogenous to the target plant cell or may be exogenous, provided that they are functional in the target plant cell.

Preferably the regulatory element is a promoter. A variety of promoters which may be employed in the vectors of the present invention are well known to those skilled in the art. Factors influencing the choice of promoter include the desired tissue specificity of the vector, and whether constitutive or inducible expression is desired and the nature of the plant cell to be transformed (eg. monocotyledon or dicotyledon). Particularly suitable constitutive promoters include the Cauliflower Mosaic Virus 35S (CaMV 35S) promoter, the maize Ubiquitin promoter, and the rice Actin promoter.

A variety of terminators which may be employed in the vectors of the present invention are also well known to those skilled in the art. The terminator may be from the same gene as the promoter sequence or a different gene. Particularly suitable terminators are polyadenylation signals, such as the CaMV 35S polyA and other terminators from the nopaline synthase (nos) and the octopine synthase (ocs) genes.

The vector, in addition to the regulatory element, the nucleic acid or nucleic acid fragment of the present invention and the terminator, may include further elements necessary for expression of the nucleic acid or nucleic acid fragment, in different combinations, for example vector backbone, origin of replication (ori), multiple cloning sites, spacer sequences, enhancers, introns (such as the maize Ubiquitin Ubi intron), antibiotic resistance genes and other selectable marker genes [such as the neomycin phosphotransferase (npt2) gene, the hygromycin phosphotransferase (hph) gene, the phosphinothricin acetyltransferase (bar or pat) gene], and reporter genes (such as beta-glucuronidase (GUS) gene (gusA)]. The vector may also contain a ribosome binding site for translation initiation. The vector may also include appropriate sequences for amplifying expression.

As an alternative to use of a selectable marker gene to provide a phenotypic trait for selection of transformed host cells, the presence of the vector in transformed cells may be determined by other techniques well known in the art, such as PCR (polymerase chain reaction), Southern blot hybridisation analysis, histochemical GUS assays, northern and Western blot hybridisation analyses.

Those skilled in the art will appreciate that the various components of the vector are operatively linked, so as to result in expression of said nucleic acid or nucleic acid fragment. Techniques for operatively linking the components of the vector of the present invention are well known to those skilled in the art. Such techniques include the use of linkers, such as synthetic linkers, for example including one or more restriction enzyme sites.

The constructs and vectors of the present invention may be incorporated into a variety of plants, including monocotyledons (such as grasses from the genera Lolium, Festuca, Paspalum, Pennisetum, Panicum and other forage and turfgrasses, corn, oat, sugarcane, wheat and barley), dicotyledons (such as arabidopsis, tobacco, white clover, red clover, subterranean clover, alfalfa, eucalyptus, potato, sugarbeet) and gym nosperms. In a preferred embodiment, the constructs and vectors may be used to transform monocotyledons, preferably grass species such as ryegrasses (Lolium species) and fescues (Festuca species) and cereals such as maize (Zea mays) and rice (Oryza sativa), more preferably perennial ryegrass, including forage- and turf-type cultivars.

In an alternate preferred embodiment, the constructs and vectors may be used to transform dicotyledons, preferably forage legume species such as clovers (Trifolium species) and medics (Medicago species), more preferably white clover (Trifolium repens), red clover (Trifolium pretense), subterranean clover (Trifolium subterraneum) and lucerne (Medicago sativa).

Techniques for incorporating the constructs and vectors of the present invention into plant cells (for example by transduction, transfection or transformation) are well known to those skilled in the art. Such techniques include Agrobacterium mediated introduction, electroporation to tissues, cells and protoplasts, protoplast fusion, injection into reproductive organs, injection into immature embryos and high velocity projectile introduction to cells, tissues, calli, immature and mature embryos. The choice of technique will depend largely on the type of plant to be transformed.

Cells incorporating the constructs and vectors of the present invention may be selected, as described above, and then cultured in an appropriate medium to regenerate transformed plants, using techniques well known in the art. The culture conditions, such as temperature, pH and the like, will be apparent to the person skilled in the art. The resulting plants may be reproduced, either sexually or asexually, using methods well known in the art, to produce successive generations of transformed plants.

In a further aspect of the present invention there is provided a plant cell, plant, plant seed or other plant part, including, e.g. transformed with, a construct or a vector of the present invention.

The plant cell, plant, plant seed or other plant part may be from any suitable species, including monocotyledons, dicotyledons and gymnosperms. In a preferred embodiment the plant cell, plant, plant seed or other plant part may be from a monocotyledon, preferably a grass or cereal species, more preferably a ryegrass (Lolium species) or fescue (Festuca species) or maize (Zea mays) or rice (Oryza sativa), even more preferably a ryegrass, most preferably perennial ryegrass, including both forage- and turf-type cultivars.

In an alternate preferred embodiment the plant cell, plant, plant seed or other plant part may be from a dicotyledon, preferably forage legume species such as clovers (Trifolium species) and medics (Medicago species), more preferably white clover (Trifolium repens), red clover (Trifolium pratense), subterranean clover (Trifolium subterraneum) and lucerne (Medicago sativa).

The present invention also provides a plant, plant seed or other plant part derived from a plant cell of the present invention.

The present invention also provides a plant, plant seed or other plant part derived from a plant of the present invention.

In a further aspect of the present invention there is provided a method of modifying the control of plant life cycles and/or growth phases, including flowering processes, flowering, plant architecture, inflorescence or flower development, in a plant, said method including introducing into said plant an effective amount of a nucleic acid or nucleic acid fragment, a construct and/or a vector according to the present invention.

By “an effective amount” it is meant an amount sufficient to result in an identifiable phenotypic trait in said plant, or a plant, plant seed or other plant part derived therefrom. Such amounts can be readily determined by an appropriately skilled person, taking into account the type of plant, the route of administration and other relevant factors. Such a person will readily be able to determine a suitable amount and method of administration. See, for example, Maniatis et al, Molecular Cloning: A Laboratory Manual, Cold Spring Harbor Laboratory, Cold Spring Harbor, the entire disclosure of which is incorporated herein by reference.

Using the methods and materials of the present invention, plant life cycles and/or growth phases, including flowering processes, flowering, plant architecture, inflorescence or flower development may be increased, decreased or otherwise modified. For example, the number of leaves produced before flowering, the number of floral organs, the number of branches, the plant stature, the number of phytomers, the number of inflorescences and flowers, may be increased, decreased or otherwise modified. They may be increased or decreased, for example, by incorporating additional copies of a sense nucleic acid of the present invention or by incorporating an antisense nucleic acid of the present invention, respectively.

The present invention will now be more fully described with reference to the accompanying Examples and drawings. It should be understood, however, that the description following is illustrative only and should not be taken in any way as a restriction on the generality of the invention described above.

In the Figures

FIG. 1 shows the nucleotide sequence of Lpld1 (Sequence ID No: 1) and corresponding deduced amino acid sequence (Sequence ID No: 2).

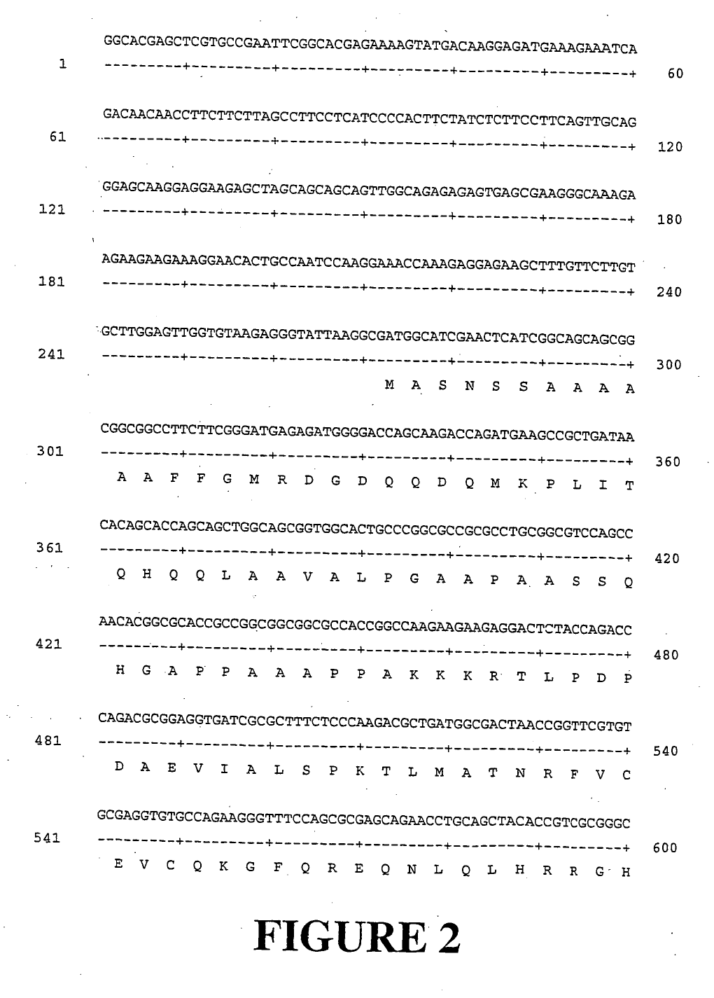

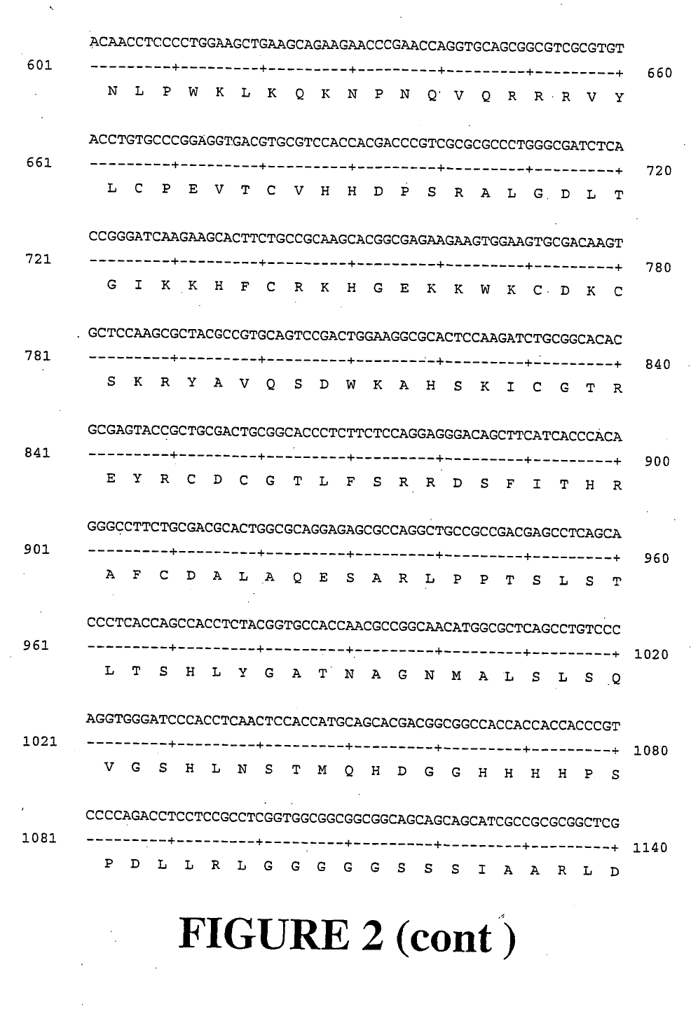

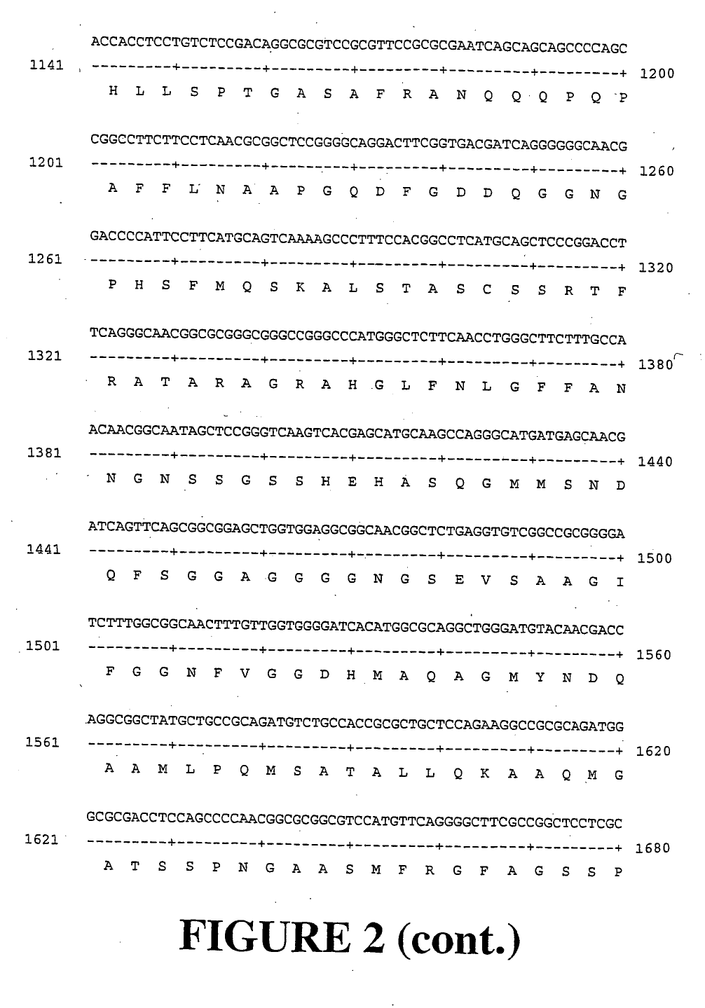

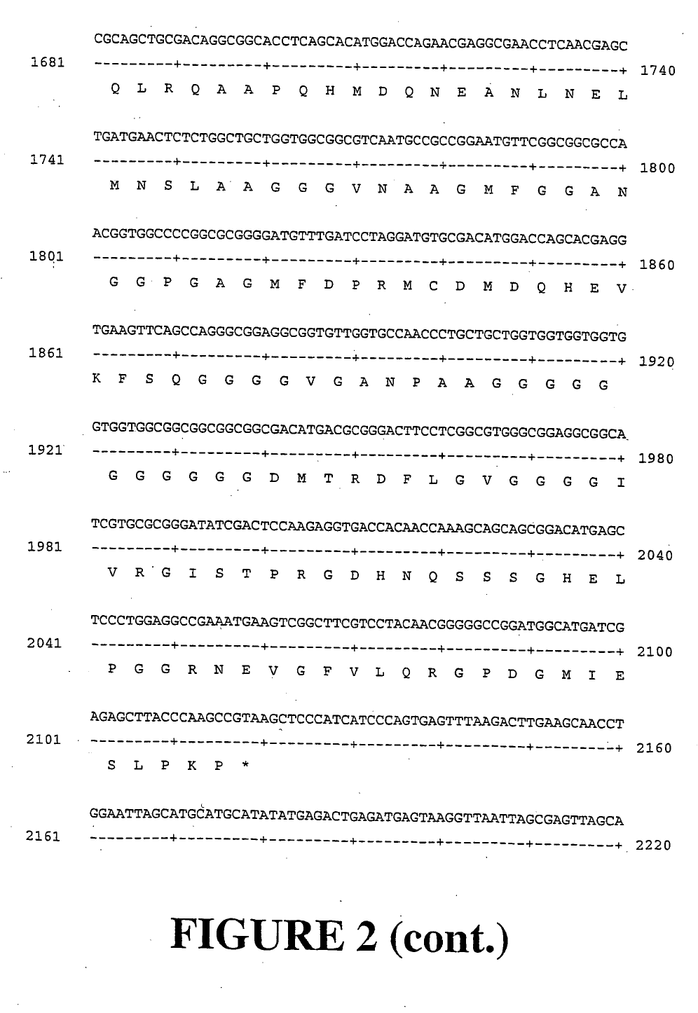

FIG. 2 shows the nucleotide sequence of Lpld2 (Sequence ID No: 3) and corresponding deduced amino acid sequence (Sequence ID No: 4).

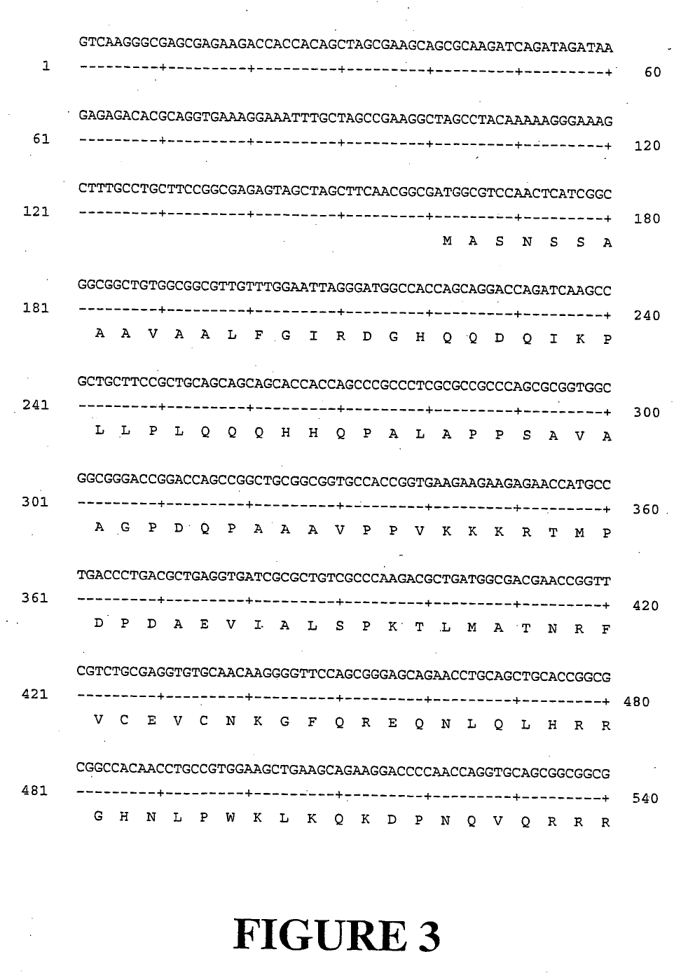

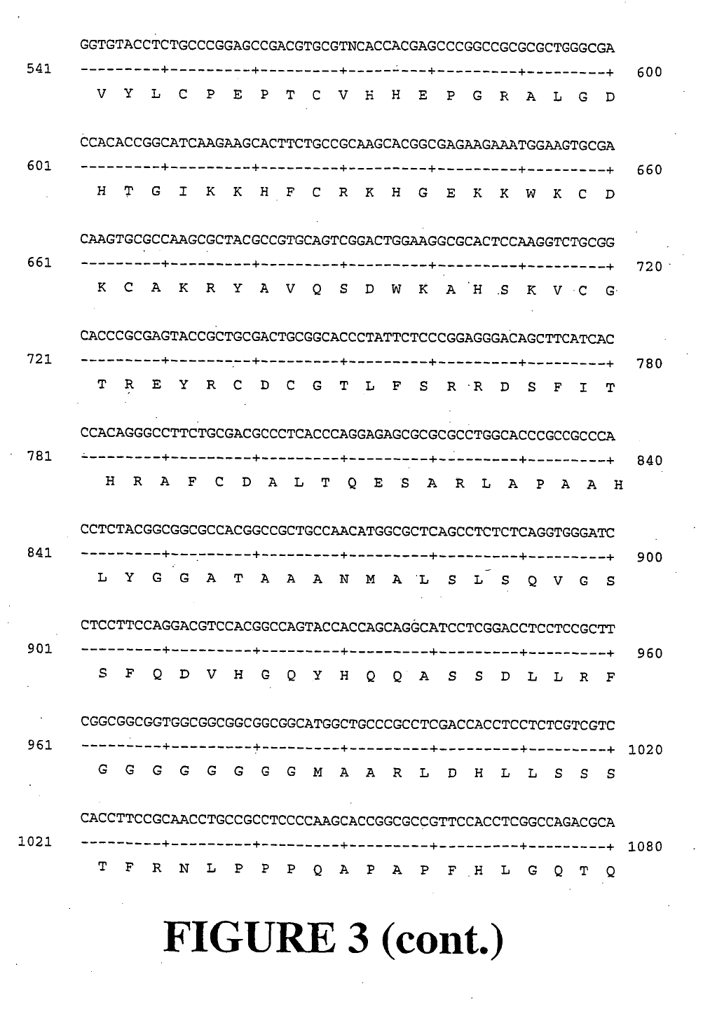

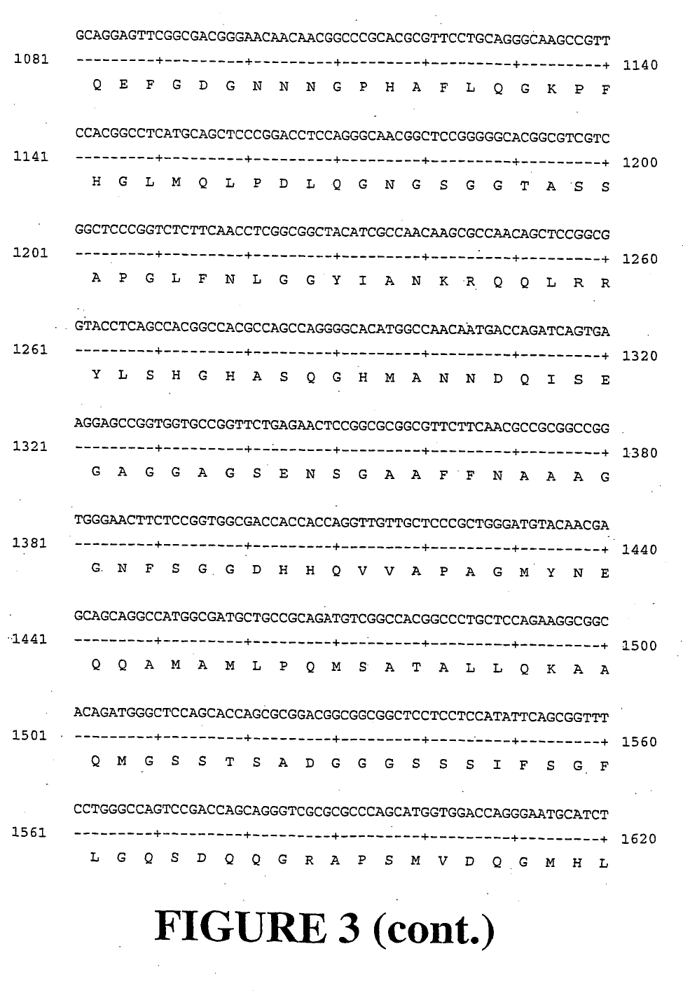

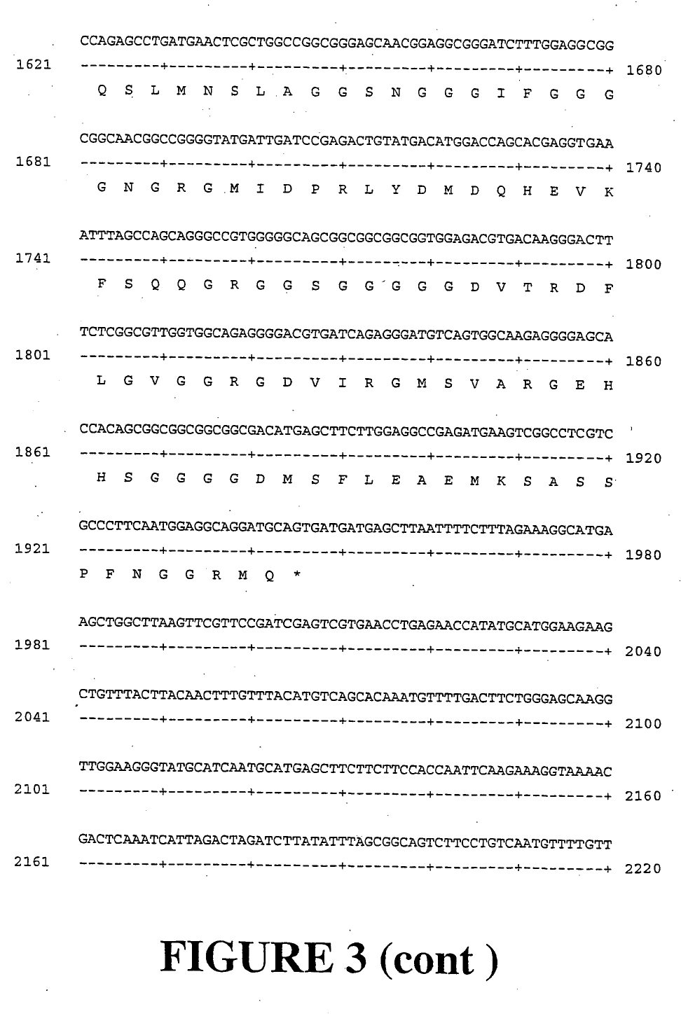

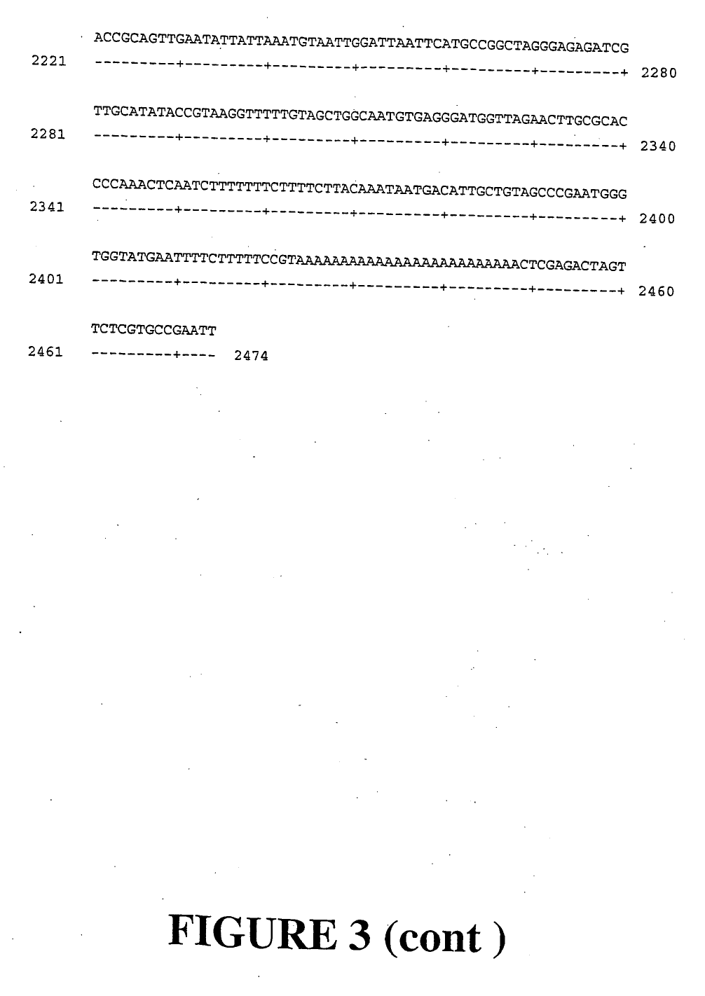

FIG. 3 shows the nucleotide sequence of Lpld3 (Sequence ID No: 5) and corresponding deduced amino acid sequence (Sequence ID No: 6).

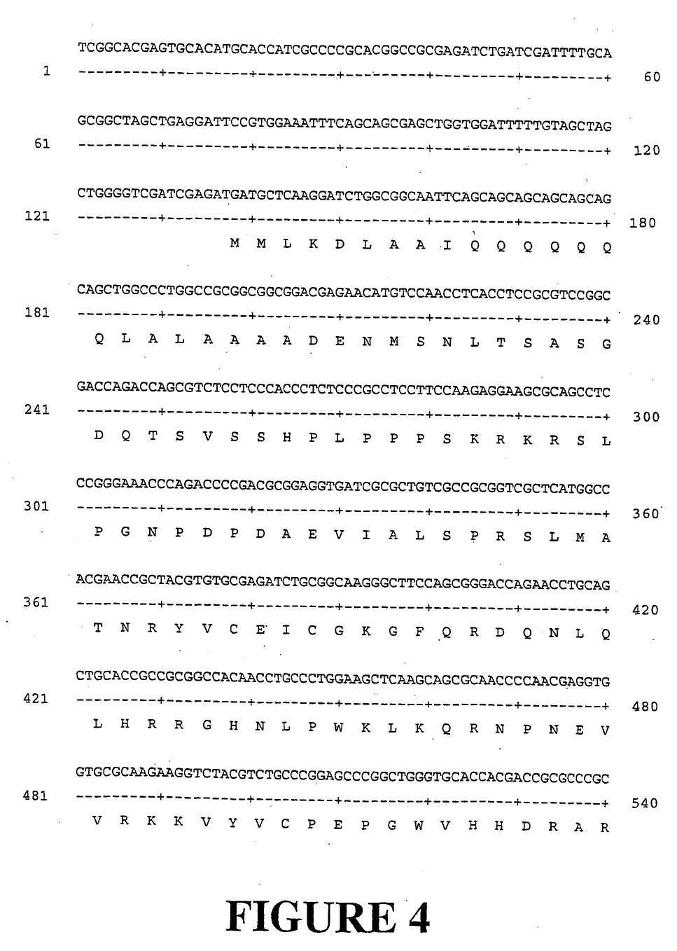

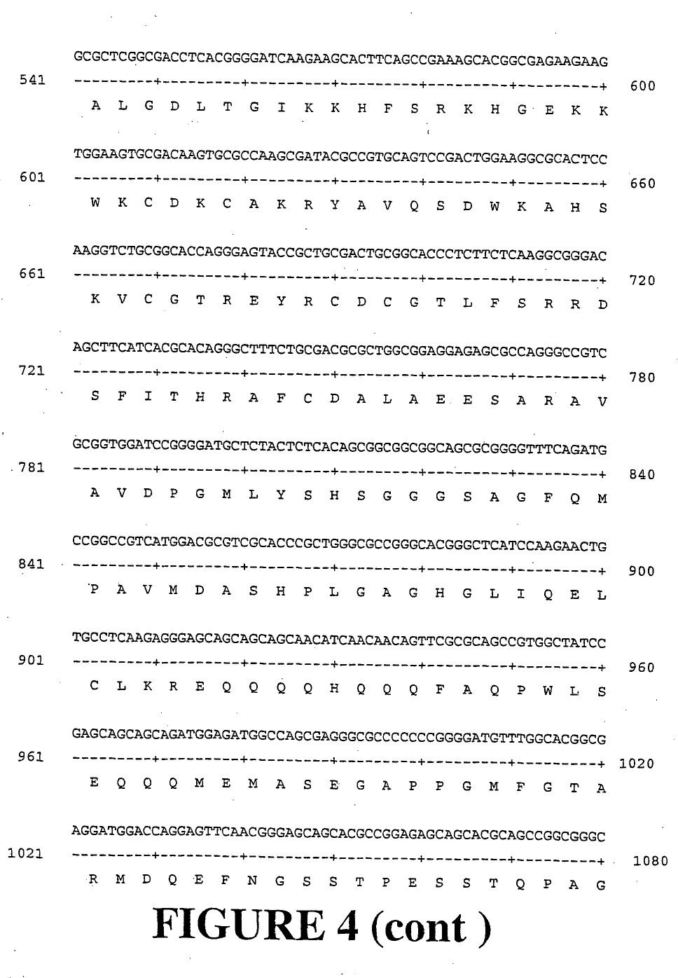

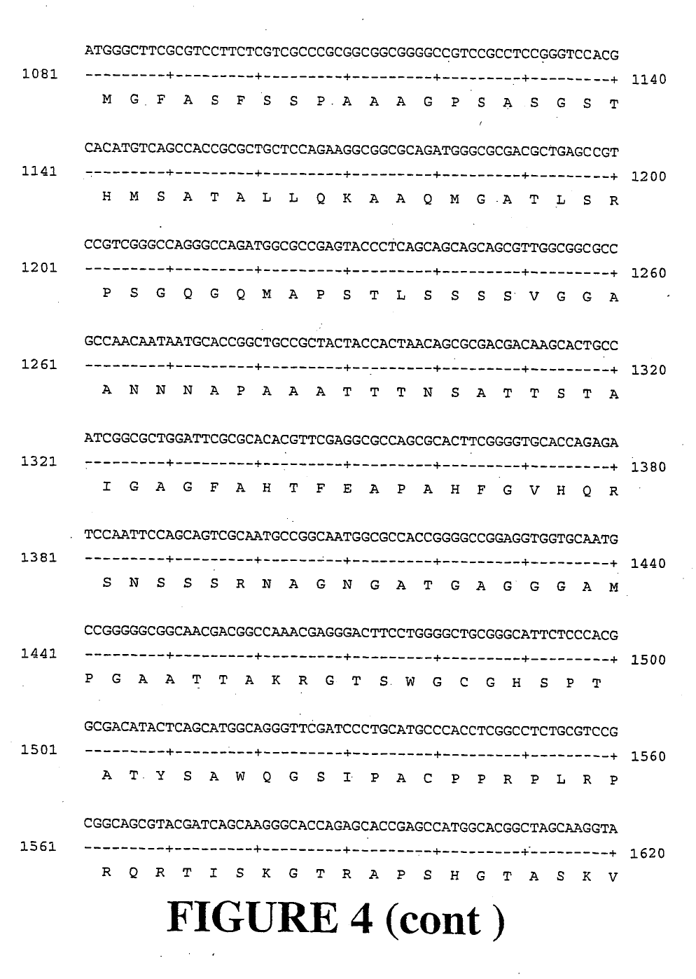

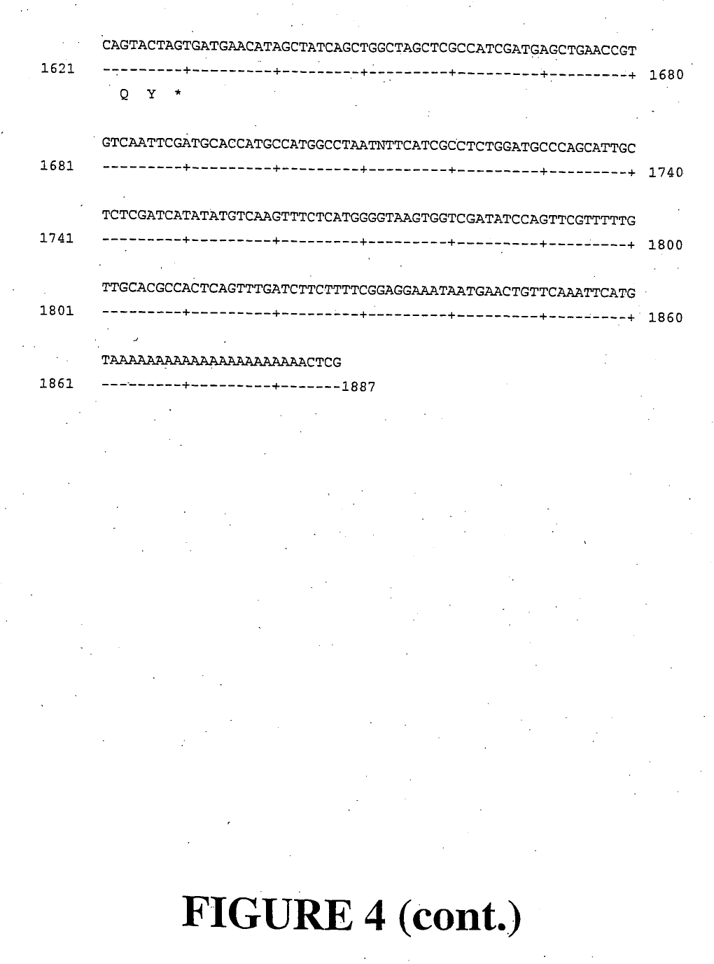

FIG. 4 shows the nucleotide sequence of Lpld4 (Sequence ID No: 7) and corresponding deduced amino acid sequence-(Sequence ID No: 8).

FIG. 5 shows a nucleotide sequence alignment of the Lpld1, Lpld2, Lpld3 and Lpld4 nucleotide sequences (Sequence ID Nos: 1, 3, 5 and 7, respectively) with conserved nucleotide positions (marked with black background) and SNPs and sequence differences (marked with white background).

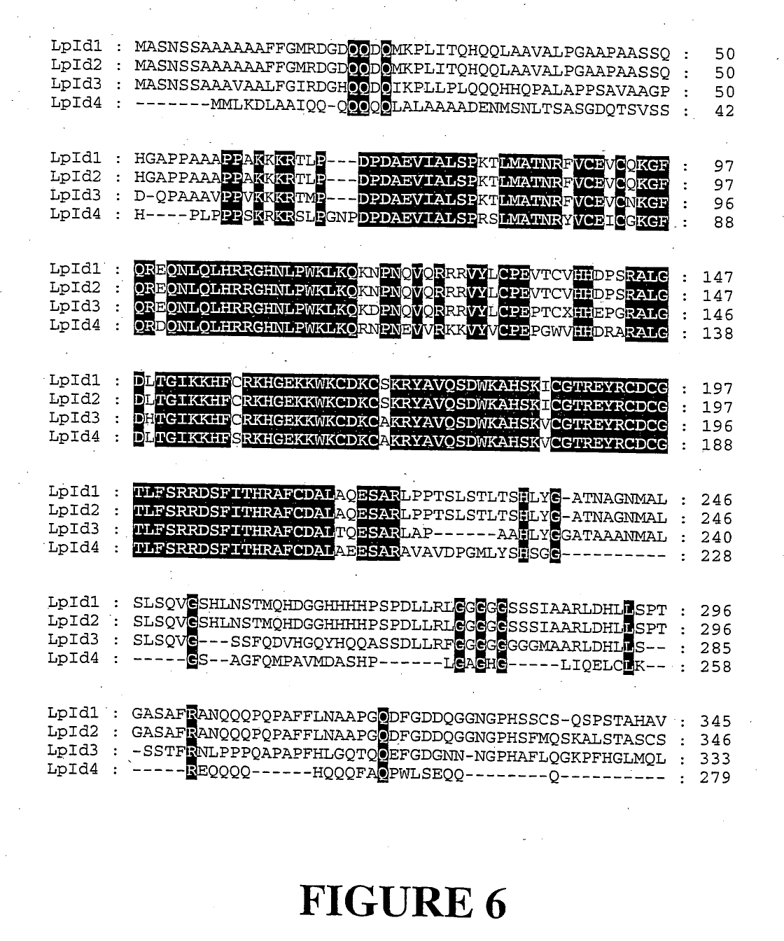

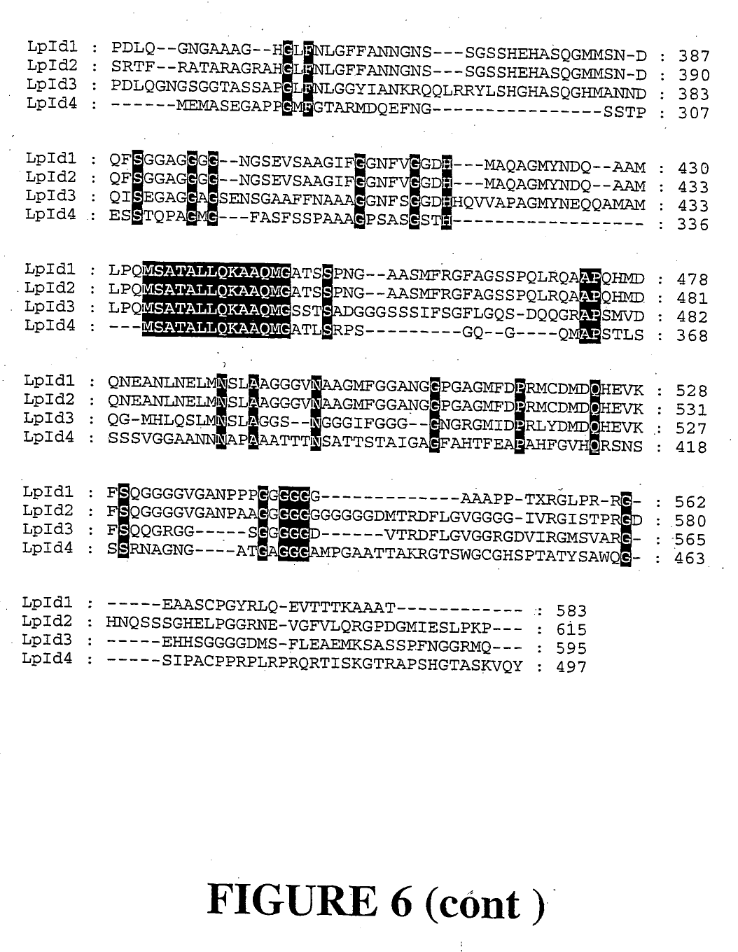

FIG. 6 shows the alignment of deduced amino acid sequences (Sequence ID Nos: 2, 4, 6 and 8, respectively) of the Lpld1, Lpld2, Lpld3 and Lpld4 nucleotide sequences with conserved amino acid residues (marked with black background) and two conserved Zinc Finger domains of transcriptional activators.

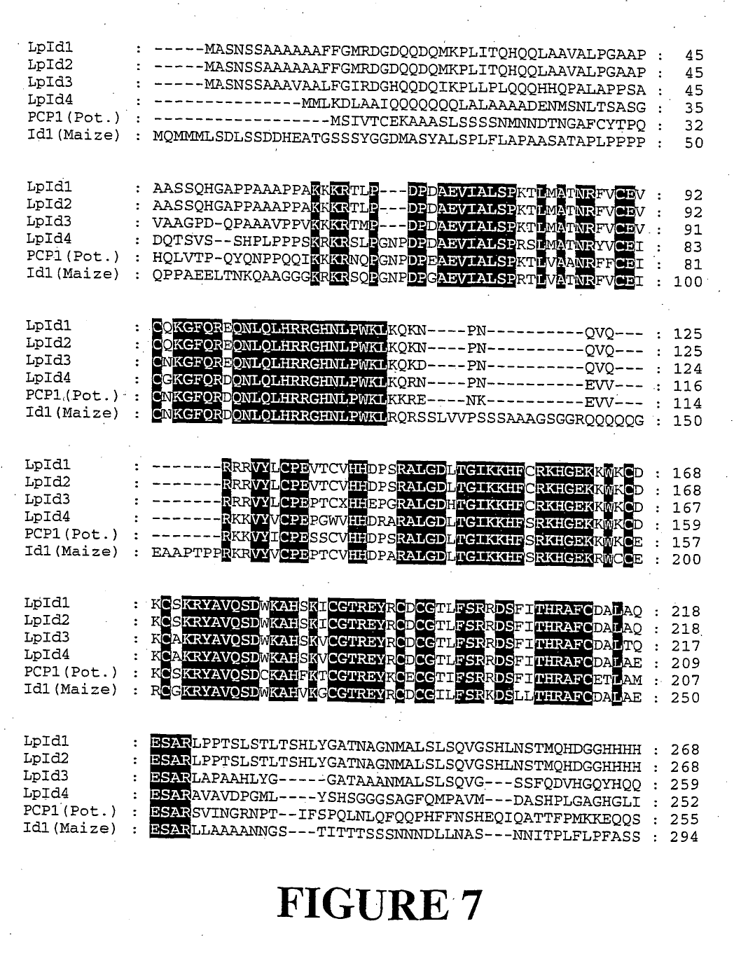

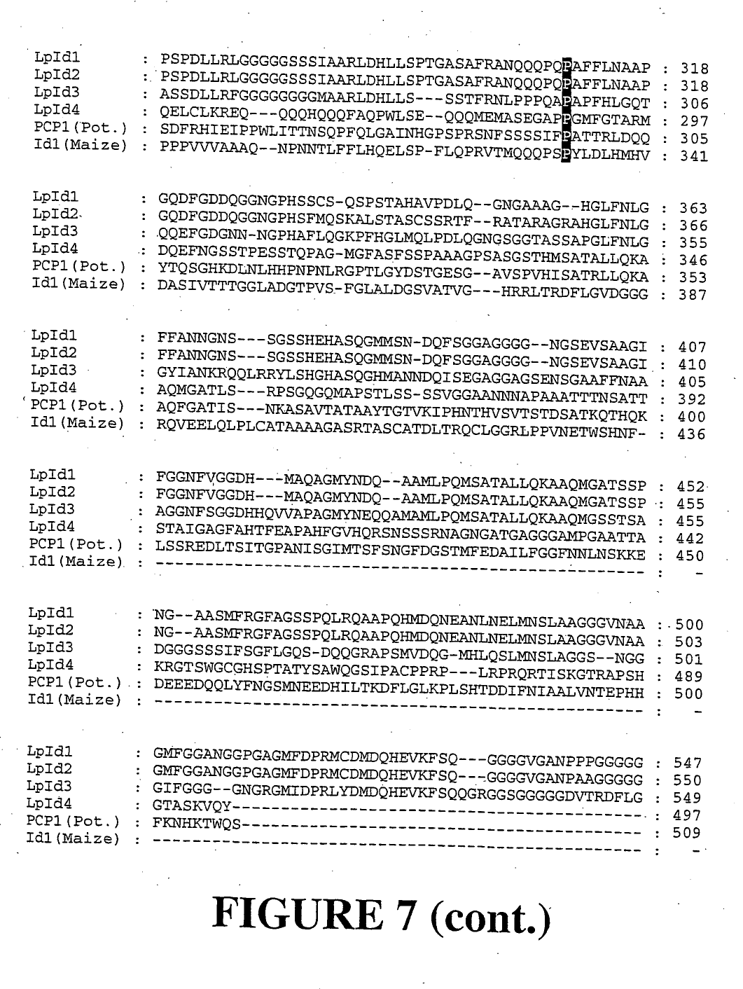

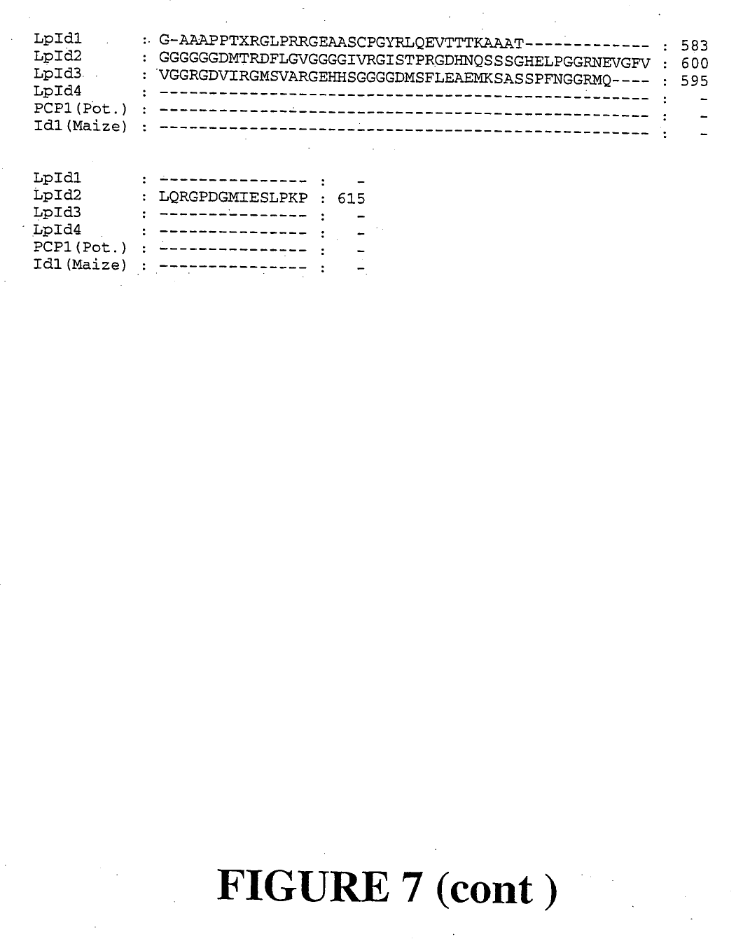

FIG. 7 shows the alignment of deduced amino acid sequences (Sequence ID Nos: 2, 4, 6 and 8, respectively) from the Lpld1, Lpld2, Lpld3 and Lpld4 nucleotide sequences with conserved amino acid residues (marked with black background) and the maize Id1 (Sequence ID No: 9) and the potato ID1-like protein PCP1 (Sequence ID No: 10).

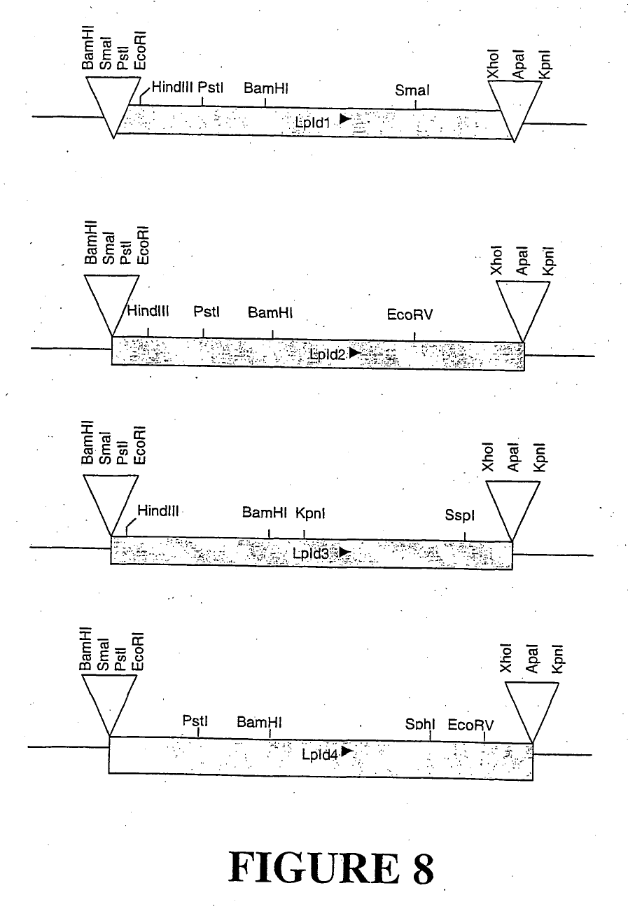

FIG. 8 shows plasmid maps of ID1 homologue cDNAs Lpld1, Lpld2, Lpld3 and Lpld4 isolated from perennial ryegrass (Lolium perenne).

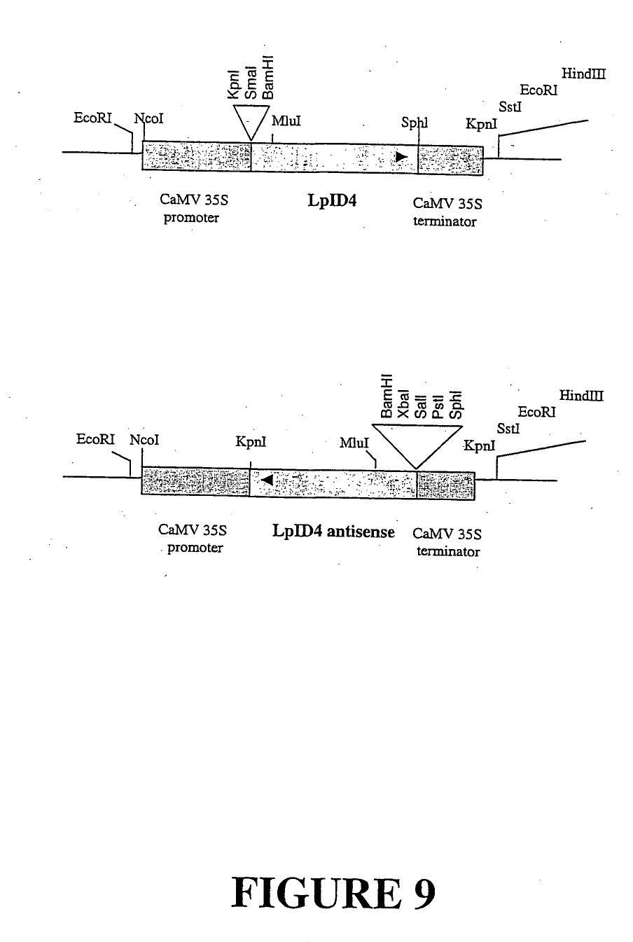

FIG. 9 shows plasmid maps of plant transformation vectors with perennial ryegrass ID1 homologue cDNA Lpld4 sequences in sense and antisense orientation under control of CaMV 35S promoter.

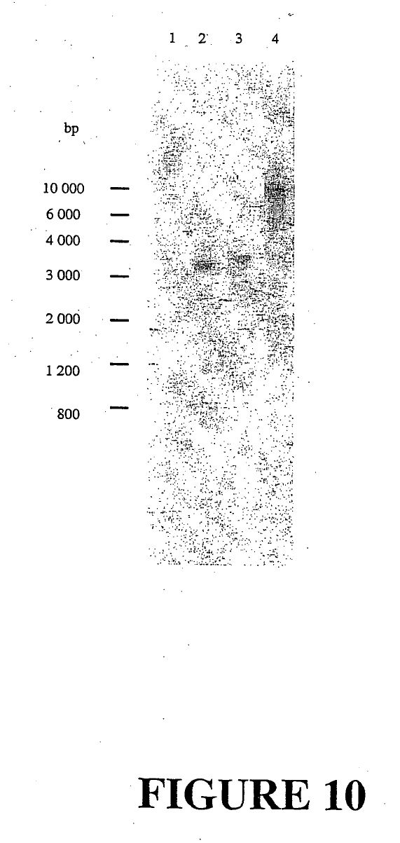

FIG. 10 shows Southern hybridisation analysis of perennial ryegrass genomic DNA using ryegrass ID1 homologue cDNA Lpld4 as hybridisation probe (Lane 1. uncut genomic DNA; lane 2. EcoRI digested genomic DNA; lane 3. HindIII digested genomic DNA; lane 4. KpnI digested genomic DNA).

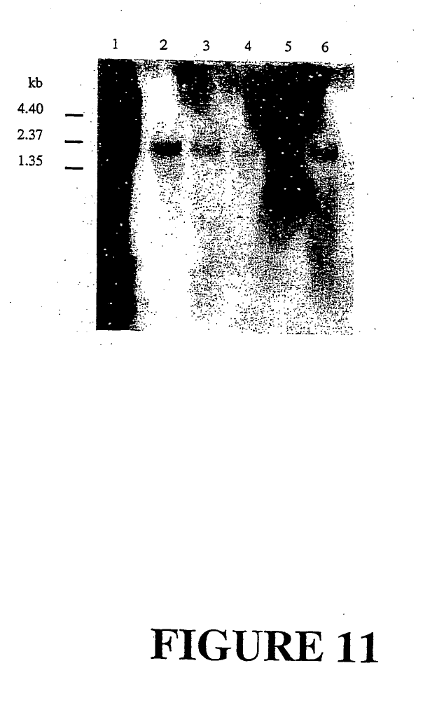

FIG. 11 shows northern hybridisation analysis revealing expression patterns of perennial ryegrass ID1 homologue cDNA Lpld4 in different perennial ryegrass plant organs and developmental stages (Lane 1. 3 day old shoots; lane 2. 3 day old roots; lane 3. 10 day old shoots; lane 4. 10 day old roots; lane 5. mature leaves; lane 6. leaves from flowering stem).

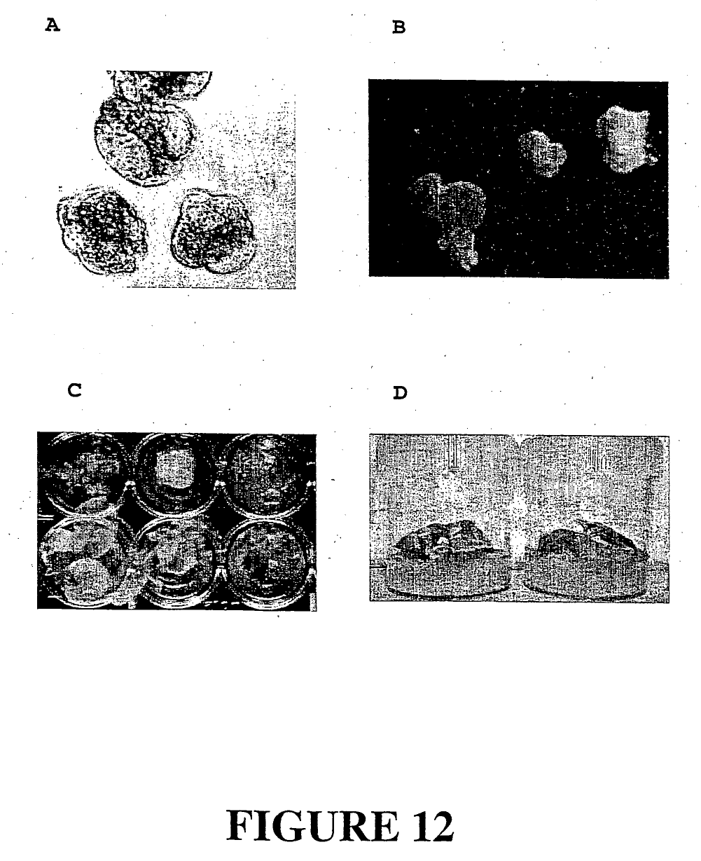

FIG. 12 shows the regeneration of transgenic tobacco plants carrying chimeric sense and antisense perennial ryegrass ID1 homologue genes.

### EXAMPLE 1

A perennial ryegrass (Lolium perenne) cDNA library was prepared from mRNA isolated from 8-10 day old seedlings. Total RNA was isolated using the Trizol method (Gibco-BRL, USA) following the manufacturers' instructions. A cDNA library was generated using the UniZAP-cDNA^{R} Synthesis Kit according to the manufacturer's protocol (Stratagene Cloning Systems, La Jolla, Calif., USA). 50,000 plaques were screened with a ryegrass ID1 PCR fragment generated using oligonucleotides designed to the maize id1 gene. Positive plaques were selected and converted into plasmids according to the protocol provided by Stratagene. Upon conversion, cDNA inserts were contained in the plasmid vector pBluescript (FIG. 8). Plasmid DNA was prepared (Qiagen, Germany) according to the protocol provided by Qiagen and cDNA inserts sequenced using dye-terminator sequencing reactions and analyzed using an Applied Biosystems ABI 3700 sequence analyser.

### EXAMPLE 2

DNA and Protein Sequence Analyses

The cDNA clones encoding ID1 proteins were identified by conducting BLAST (Basic Local Alignment Search Tool; Altschul et al. (1993) J. Mol, Biol. 215:403-410) searches. The cDNA sequences obtained were analysed for similarity to all publicly available DNA sequences contained in the ANGIS nucleotide database using the BLASTN algorithm provided by the National Center for Biotechnology Information (NCBI). The DNA sequences were translated in all reading frames and compared for similarity to all publicly available protein sequences contained in the SWISS-PROT protein sequence database using BLASTx algorithm (v 2.0.1) (Gish and States (1993) Nature Genetics 3:266-272) provided by the NCBI. The results from the deduced amino acid sequence alignments are shown on FIG. 7.

### EXAMPLE 3

Development of Transformation Vectors Containing Chimeric Genes with ID1 cDNA Sequences from Perennial Ryegrass

To alter the expression of ID1 gene activity in transgenic plants through antisense and/or sense suppression technology and for, over-expression or ectopic a set of sense and antisense transformation vectors was produced.

cDNA fragments were generated from the cDNA clone of Lpld4 using the restriction enzymes BamHI and SphI. The sense construct was, generated by direct cloning of this fragment into the transformation vector pDH51, which was digested with the same enzymes. The antisense construct was obtained by removing the 3′ overhang following SphI digestion to produce a blunt end. The fragment was then digested with BamHI and this fragment was cloned into pDH51 digested with BamHI and SmaI. Transformation vectors containing this 750 bp region of the Lpld4 cDNA in sense and antisense orientations under-the control of the CaMV 35S promoter were generated (FIG. 9).

### EXAMPLE 4

Production of Transgenic Tobacco Plants Carrying Chimeric ID1 Homologue Genes from Perennial Ryegrass

A set of transgenic tobacco plants carrying chimeric sense and antisense ID1 homologue genes from perennial ryegrass were produced.

pDH51-based transformation vectors with Lpld4 cDNA comprising a 750 bp fragment in sense and antisense orientations under the control of the CaMV 35S promoter were generated (FIG. 9).

Direct gene transfer experiments to tobacco protoplasts were performed using these transformation vectors (Table 1).

TABLE 1
Production of transgenic tobacco calli carrying chimeric perennial ryegrass
ID1 homologue genes (in sense and antisense orientation) from direct gene
transfer to protoplasts

| | Transfected | transformed | transformation |
|---|---|---|---|
| Construct | protoplasts | calli | efficiency |
| pLpld4 | 1.2 × 10⁶ | 46 | 0.38 × 10⁻⁵ |
| sense | | | |
| pLpld4 | 1.2 × 10⁶ | 58 | 0.48 × 10⁻⁵ |
| antisense | | | |

The production of transgenic tobacco plants carrying the perennial ryegrass Lpld4 cDNA under the control of the constitutive CaMV 35S promoter is described here in detail.

Isolation of Mesophyll Protoplasts from Tobacco Shoot Cultures

2 to 4 fully expanded leaves of a 6 week-old shoot culture were placed under sterile conditions (work in laminar flow hood, use sterilized forceps, scalpel and blades) in a 9 cm plastic culture dish containing 12 ml enzyme solution [1.0% (w/v) cellulase “Onozuka” R10 and 1.0% (w/v) Macerozyme® R10]. The leaves were wetted thoroughly with enzyme solution and the mid-ribs removed. The leaf halves were cut into small pieces and incubated overnight (14 to 18 h) at 25° C. in the dark without shaking

The protoplasts were released by gently pipetting up and down, and the suspension poured through a 100 μm stainless steel mesh sieve on a 100 ml glass beaker. The protoplast suspension was mixed gently, distributed into two 14 ml sterile plastic centrifuge tubes and carefully overlayed with 1 ml W5 solution. After centrifugation for 5 min. at 70 g (Clements Orbital 500 bench centrifuge, swing-out rotor, 400 rpm), the protoplasts were collected from the interphase and transferred to one new 14 ml centrifuge tube. 10 ml W5 solution were added; the protoplasts resuspended by gentle tilting the capped tube and pelleted as before. The protoplasts were resuspended in 5 to 10 ml W5 solution and the yield determined by counting a 1:10 dilution in a haemocytometer.

Direct Gene Transfer to Protoplasts Using Polyethylene Glycol

The protoplasts were pelleted [70 g (Clements Orbital 500 bench centrifuge, 400 rpm) for 5 min.] and resuspended in transformation buffer to a density of 1.6×10 protoplasts/ml. Care should be taken to carry over as little as possible W5 solution into the transformation mix. 300 μl samples of the protoplast suspension (ca. 5×10⁵ protoplasts) were aliquotted in 14 ml sterile plastic centrifuge tubes, 30 μl of transforming DNA were added. After carefully mixing, 300 μl of PEG solution were added and mixed again by careful shaking. The transformation mix was incubated for 15 min. at room temperature with occasional shaking. 10 ml W5 solution were gradually added, the protoplasts pelleted [70 g (Clements Orbital 500 bench centrifuge, 400 rpm) for 5 min.] and the supernatant removed. The protoplasts were resuspended in 0.5 ml K3 medium and ready for cultivation.

Culture of Protoplasts, Selection of Transformed Lines and Regeneration of Transgenic Tobacco Plants

Approximately 5×10⁵ protoplasts were placed in a 6 cm petri dish. 4.5 ml of a pre-warmed (melted and kept in a water bath at 40 to 45° C.) 1:1 mix of K3:H medium containing 0.6% SeaPlaque™ agarose were added and, after gentle mixing, allowed to set.

After 20 to 30 min the dishes were sealed with Parafilm® and the protoplasts were cultured for 24 h in darkness at 24° C., followed by 6 to 8 days in continuous dim light (5 μmol m⁻² s⁻¹, Osram L36 W/21 Lumilux white tubes), where first and multiple cell divisions occur. The agarose containing the dividing protoplasts was cut into quadrants and placed in 20 ml of A medium in a 250 ml plastic culture vessel. The corresponding selection agent-was added to the final concentration of 50 mg/l kanamycin sulphate (for npt2 expression) or 25 mg/l hygromycin B (for hph expression) or 20 mg/l phosphinotricin (for bar expression). Samples were incubated on a rotary shaker with 80 rpm and 1.25 cm throw at 24° C. in continuous dim light.

Resistant colonies were first seen 3 to 4 weeks after protoplast plating, and after a total time of 6 to 8 weeks protoplast-derived resistant colonies (when 2 to 3, mm in diameter) were transferred onto MS morpho medium solidified with 0.6% (w/v) agarose in 12-well plates and kept for the following 1 to 2 weeks at 24° C. in continuous dim light (5 μmol m⁻² s⁻¹, Osram L36 W/21 Lumilux white tubes), where calli proliferated, reached a size of 8 to 10 mm, differentiated shoots that were rooted on MS hormone free medium leading to the recovery of transgenic tobacco plants (Table 1 and FIG. 12).

### EXAMPLE 5

Genomic Organization of Perennial Ryegrass ID1 Homologue Genes

Genomic DNA from perennial ryegrass was digested with the following restriction enzymes EcoRI, HindIII and KpnI. Southern blot analysis was then performed according to standard protocols (Ausubel et al. (1994) Current Protocols in Molecular Biology, Greene Publishing Associates and Wiley Interscience). The probe used for screening this blot was a 750 bp gene specific fragment of the Lpld4 cDNA obtained by restriction digestion with BamHI and SphI. Lpld4 exists as a single copy gene in the genome of perennial ryegrass (FIG. 10).

### EXAMPLE 6

Expression of Perennial Ryegrass ID1 Homologue Genes

A northern hybridisation analysis with RNA samples isolated from perennial ryegrass at different developmental stages was performed to determine patterns of organ and developmental expression of ryegrass ID1 genes. Total RNA was extracted from the following tissues using Trizol reagent (GibcoBRL, USA) three day old shoots and roots, ten day old shoots and roots, mature leaves and leaves taken from the flowering stem. Northern blot analysis was performed according to according to standard protocols (Ausubel et al. (1994) Current Protocols in Molecular Biology, Greene Publishing Associates and Wiley Interscience). The probe used for screening this blot was a 750 bp gene specific fragment of the Lpld4 cDNA obtained by restriction digestion with BamHI and SphI. Lpld4 is most strongly expressed in three-day old roots and mature leaves (FIG. 11).

Finally, it is to be understood that various alterations, modifications and/or additions may be made without departing from the spirit of the present invention as outlined herein.

It will also be understood that the term “comprises” (or its grammatical variants) as used in this specification is equivalent to the term “includes” and should not be taken as excluding the presence of other elements or features.

Documents cited in this specification are for reference purposes only and their inclusion is not an acknowledgment that they form part of the common general knowledge in the relevant art.

1. A substantially purified or isolated nucleic acid or nucleic acid fragment encoding an amino acid sequence for an ID1 protein from a ryegrass (Lolium) or fescue (Festuca) species, or a functionally active fragment or variant thereof.

2. A nucleic acid or nucleic acid fragment according to claim 1, wherein said ryegrass is perennial ryegrass (Lolium perenne).

3. A nucleic acid or nucleic acid fragment according to claim 1, including a nucleotide sequence selected from the group consisting of (a) sequences shown in FIGS. 1, 2, 3, 4 and 5 hereto (Sequence ID Nos: 1, 3, 5 and 7); (b) complements of the sequences in (a); (c) sequences antisense to the sequences recited in (a) and (b); and (d) functionally active fragments and variants of the sequences recited in (a), (b) and (c).

4. A construct including a nucleic acid or nucleic acid fragment according to claim 1.

5. A vector including a nucleic acid or nucleic acid fragment according to claim 1.

6. A vector according to claim 5, further including a promoter and a terminator, said promoter, nucleic acid or nucleic acid fragment and terminator being operatively linked.

7. A plant cell, plant, plant seed or other plant part, including a construct according to claim 4 or a vector according to claim 5.

8. A plant, plant seed or other plant part derived from a plant cell or plant according to claim 7.

9. A method of modifying plant life cycles and/or growth phases in a plant, said method including introducing into said plant an effective amount of a nucleic acid or nucleic acid fragment according to claim 1, a construct according to claim. 4, and/or a vector according to claim 5.

10. A method according to claim 9 wherein said plant life cycle and/or growth phase is selected from the group consisting of flowering processes, flowering and plant architecture, and inflorescence and flower development.

11. Use of a nucleic acid or nucleic acid fragment according to claim 1, and/or nucleotide sequence information thereof, and/or single nucleotide polymorphisms thereof as a molecular genetic marker.

12. A substantially purified or isolated polypeptide from a ryegrass (Lolium) or fescue (Festuca) species, selected from the group consisting of ID1 and ID1-like proteins; and functionally active fragments and variants thereof.

13. A polypeptide according to claim 12, wherein said ryegrass is perennial ryegrass (Lolium perenne).

14. A polypeptide according to claim 12, wherein said polypeptide includes an amino acid sequence selected from the group of sequences shown in FIGS. 1, 2, 3, 4 and 6 hereto (Sequence. ID Nos: 2, 4, 6 and 8); and functionally active fragments and variants thereof.
